(19) United States
(12) Patent Application Publication
(10) Pub. No.: US 2025/0367341 A1
(43) Pub. Date: Dec. 4, 2025

(54) TOUGH ADHESION WITH A TOUGH GEL AND CHITOSAN BRIDGING POLYMER

(71) Applicant: President and Fellows of Harvard College, Cambridge, MA (US)

(72) Inventors: Benjamin Ross Freedman, Brookline, MA (US); David J. Mooney, Cambridge, MA (US)

(21) Appl. No.: 18/869,604

(22) PCT Filed: May 30, 2023
(86) PCT No.: PCT/US2023/023815

§ 371 Date: Nov. 26, 2024

Related U.S. Application Data
(60) Provisional application No. 63/347,252, filed on May 31, 2022.
(60) Provisional application No. 63/414,964, filed on Oct. 11, 2022.

(51) Int. Cl. A61L 24/00; A61L 24/04

(52) U.S. Cl. CPC A61L 24/0031; A61L 24/043

(57) ABSTRACT

The present disclosure shows that by combining pH-responsive bridging chitosan polymer chains and a tough hydrogel dissipative matrix one can achieve unprecedented ultra-tough adhesion to tissues (>2000J/m²) in 5-10 mins without covalent bond formation. The strong non-covalent adhesion was shown to be stable under physiologically relevant conditions and strongly influenced by chitosan molecular weight. molecular weight of polymers in the matrix, and pH. The adhesion mechanism relies primarily on the topological entanglement between the chitosan chains and the permeable adherends. The present disclosure also discloses dry polymer films to generate instant adhesion between hydrogel-hydrogel and hydrogel-elastomer surfaces. Unprecedented adhesive energies (>3000J/m²) between alginate-polyacrylamide tough hydrogels were achieved instantaneously using an intermediate chitosan film, governed by pH change, H-bonding, and bridging polymer entanglement. Furthermore, this strategy also generates instant strong adhesion between acrylic elastomers and tough hydrogels with adhesion energy as high as 4000J/m².

### RELATED APPLICATIONS

This application claims priority to U.S. Provisional Application No. 63/347,252, filed on May 31, 2022, and U.S. Provisional Application No. 63/414,964, filed on Oct. 11, 2022. The entire contents of each of the foregoing applications are expressly incorporated herein by reference.

### GOVERNMENT SUPPORT

This invention was made with government support under AG065495 awarded by National Instituted of Health (NIH). The government has certain rights in this invention.

### BACKGROUND

Tissue adhesives have received increased attention in the last decades because of their potential applications as sealants,^{[1,2]} wound dressings,^{[3]} and drug delivery systems,^{[4, 5]} among others.^{[6, 7]} Tissue adhesives approved for use inside the body are indicated as an adjunct to suturing and staples, but one day may replace, or at least be a viable alternative, to traditional staples and sutures.^{[2]} Recently, advances in hydrogel technologies^{[8, 9]} have highlighted their versatility, biocompatibility, and tunability as tissue adhesives. Additionally, several groups have recently highlighted the current progress in hydrogel adhesives technologies, including their applications and challenges.^{[7, 10, 11]}

Hydrogel-based adhesives, however, suffer from several limitations, including the need of strong covalent bonds directly with tissue proteins to generate adhesion.^{[11]} Cyanoacrylate-based adhesion involves the diffusion of reactive monomers at tissue sites and subsequent in-situ crosslinking, but this often releases toxic unreacted monomers into the

bloodstream or generates reactive radical species.^{[12]} Another strategy for direct covalent bond based adhesives is the use of a bridging layer and coupling reagents to facilitate bond formation between a gel and tissue. For example, carbodiimide coupling reactions combined with a tough hydrogel matrix achieve adhesion energies of more than 1000J/m².^{[1]} However, this strategy depends on covalent bond junctions and the need for specific and complementary reactive functional groups in both the hydrogel and tissue. Other systems involving direct bond formation between the gel and tissue have also been reported, achieving strong adhesion in a few minutes, but again requiring reactive species such as activated NHS-esters or aldehydes, among other reactive groups, for bond formation.^{[13, 14]} Relying on specific functional groups can be a limitation under physiological and clinically relevant environments where blood and other fluids can dilute and interfere with the chemical reactions responsible for bond and adhesion formation.^{[2]}

Tissue adhesives relying only on non-covalent bonding (˜1-50 kT for a single H-bond)^{[15]} are typically weak, fragile, and slow to adhere.^{[16, 17]} Adhesives using direct electrostatics and hydrogen bonding interactions between gel and tissue were recently reported. Non-covalent adhesion was achieved instantly but it was weak (<200J/m²).^{[17]} Topological wet adhesion between two permeable adherends is another strategy which can be achieved merely based on topology and chain entanglements between the two adherends.^{[18, 19]} As with most other adhesives relying on physical interactions, tissue adhesion was weak (<200J/m²) and time consuming (>1 h to achieve peak strength), which may make biomedical application challenging. However, the mechanism of topological adhesion remains promising as it does not rely on specific functional groups and could be further harnessed in engineering new biomaterials.

Engineering strong interfacial toughness between polymer networks would address unmet needs in tissue adhesion, biomedicine, and bioelectronics (1.1-5.1). Traditional adhesion between hydrogels primarily relies on the formation of chemical bonds between the adherends (6.1, 7.1). Recently, an adhesion strategy using a liquid chitosan bridging layer to bind hydrogels identified time and pH dependent adhesion mechanisms without the need for covalent bonding (8.1). Over time, liquid chitosan (pKa˜6.5) diffuses into the hydrogel surface (pH>6.5) and becomes deprotonated. Chitosan deprotonation results in hydrogen bonding between polymer chains, leading to their entanglement and formation of molecular interlocks between the bridging polymer and the adherend matrix, together generating unprecedented adhesion without covalent bonding.

However, a central limitation of liquid-based bridging polymers is that the strength of adhesion is time-dependent and typically requires minutes to hours to reach equilibrium (1.1, 8.1-10.1) due to the low diffusivity of macromolecules (11.1). In contrast to ‘liquid-based’ adhesion strategies, ‘dry’ adhesion relies on rapid absorption of fluids at the substrate interface and simultaneous chemical bonding, enabling instant adhesion (12.1, 13.1). Although recent studies have demonstrated fast liquid-based adhesion when coupled with hydration of a dry polymer matrix, a dual approach where adhesion is driven by both physical non-covalent interactions, such as chain entanglements and hydrogen bonding, as well as ‘dry’ adhesion strategies, such as local hydration and swelling, has not been investigated. Such a technology could enable a facile and practical method to couple different hydrogel biomaterials for diverse clinical indications. This study explores the dual mechanistic approach using non-covalent dry adhesion, presenting a simple, versatile strategy using dry polymer films to generate instant tough adhesion between hydrogel and elastomer surfaces.

### SUMMARY OF THE INVENTION

The present invention is based on the unexpected discovery that a double polymer network hydrogel can form strong adhesion with a surface (e.g., a tissue or an elastomer) within minutes through a bridging polymer and without the presence of a coupling agent. As described herein, Applicant investigated adhesion to tissues and design an alginate-polyacrylamide tough adhesive (TA) that generates ultra-tough (>2000J/m²) and unprecedented topological tissue adhesion within minutes, without the need of covalent bond formation. The TA relies on a tough double network hydrogel as an energy dissipation matrix and a bridging polymer that bonds the gel and tissue together. The bridging polymer (e.g., chitosan) disclosed herein is proposed to act as a stimuli-responsive polymer by forming strong intermolecular H-bonds upon a change in pH. This property allows the chains to diffuse and form an internal network between two permeable adherends, in this case, gel and tissue. The present disclosure also provides how the properties of the tough gel matrix and the bridging polymer, including molecular weight, viscosity, and pH influence adhesion. Furthermore, the present disclosure provides a strategy to accelerate chain diffusion and entanglement in the hydrogel to tune adhesion time and strength. Strong, rapid adhesion of >1500J/m² was generated. The strong and fast adhesion disclosed herein relies on a specific combination of mechanics and network topologies without the need for covalent bond formation (and/or through a coupling agent). Given the unprecedented adhesive properties obtained with these biomaterials, multiple biomedical applications are possible.

In a first embodiment, the present disclosure provides a tough adhesive comprising a hydrogel and a bridging polymer; wherein the hydrogel comprises a first polymer network and a second polymer network; wherein after contacting said hydrogel and said bridging polymer with a surface, said hydrogel is adhered to said surface via said bridging polymer, and an adhesion between said surface and said hydrogel is greater than or equal to 400 J/m² approximately 3 to about 10 minutes, and wherein said tough adhesive does not include a coupling agent. In a specific embodiment, the surface is a tissue. In another specific embodiment, the surface is an elastomer. In some embodiments, said first polymer network comprises covalent crosslinks and said second polymer network comprises ionic crosslinks.

In a second embodiment, for the tough adhesive of the first embodiment, the hydrogel is not covalently bound to the surface or the bridging polymer.

In a third embodiment, for the tough adhesive of the first or second embodiment, the first polymer network is a polyacrylamide polymer, the second polymer network is an alginate polymer, and the bridging polymer is a chitosan polymer.

In a fourth embodiment, for the tough adhesive of the first, second, or third embodiment, the hydrogel is dried before application to the surface.

In a fifth embodiment, for the tough adhesive of the fourth embodiment, the adhesion between the surface and the hydrogel is greater than or equal to 1000 J/m² approximately about 1 minute after contacting the dehydrated hydrogel and the bridging polymer with the surface.

In a sixth embodiment, for the tough adhesive of the third, fourth, or fifth embodiment, the alginate polymer has an average molecular weight of about 100 kDa to about 300 kDa, or wherein the alginate polymer has an average molecular weight of about 200 kDa to about 300 kDa.

In a seventh embodiment, for the tough adhesive of the third, fourth, fifth, or sixth embodiment, chitosan polymer has a molecular weight of about 100 kDa to about 600 kDa, or the chitosan polymer has a molecular weight of about 150 kDa to about 250 kDa.

In an eighth embodiment, for the tough adhesive of the first, second, third, fourth, fifth, sixth, or seventh embodiment, the bridging polymer is a chitosan solution which comprises about 1% to about 2% (weight/volume) chitosan.

In a ninth embodiment, the present disclosure provides a method of applying the tough adhesive of the first, second, third, sixth, seventh, or eighth embodiment, wherein the method comprises the steps of adding the bridging polymer to the hydrogel; and compressing the hydrogel gel with the bridging polymer onto the surface.

In a tenth embodiment, the present disclosure provides a method of applying the tough adhesive of the fourth, fifth, sixth, seventh, or eighth embodiment, wherein the method comprises the steps of drying the hydrogel; adding the bridging polymer to the dried hydrogel; and compressing the dried hydrogel gel with the bridging polymer onto the surface.

In an eleventh embodiment, for the method of the ninth or tenth embodiment, the hydrogel with the bridging polymer is compressed onto the surface for up to about 1 minute, or the hydrogel with the bridging polymer is compressed onto the surface for up to about 10 minutes.

In a twelfth embodiment, the present disclosure provides a tough adhesive comprising a hydrogel comprising a first polymer network and an optional second polymer network, and a dried bridging polymer film; wherein an adhesion between the hydrogel and the bridging polymer is greater than or equal to 150 J/m² approximately about 5 seconds after contacting the hydrogel with the dried bridging polymer film.

In a thirteenth embodiment, for the tough adhesive of the twelfth embodiment, the film is not covalently bound to the hydrogel.

In a fourteenth embodiment, for the tough adhesive of the twelfth or thirteenth embodiments, the first polymer network is a polyacrylamide polymer, the second polymer network is an alginate polymer, and the dried bridging polymer film is a dried chitosan polymer film.

In a fifteenth embodiment, for the tough adhesive of the twelfth, thirteenth, or fourteenth embodiment, the hydrogel further comprises a polyethylene mesh.

In a sixteenth embodiment, for the tough adhesive of the twelfth, thirteenth, fourteenth, or fifteenth embodiment, a first portion of the hydrogel is adhered to a second portion of the hydrogel via the bridging polymer, and wherein an adhesion between the first and second portions of the hydrogel is greater than or equal to 150 J/m² approximately about 5 seconds after contacting the first portion of hydrogel and the dried bridging polymer film with the second portion of the hydrogel.

In a seventeenth embodiment, for the tough adhesive of the twelfth, thirteenth, fourteenth, or fifteenth embodiment, the tough adhesive further comprises a first and second hydrogel which each comprise a first polymer network and an optional second polymer network; wherein the first hydrogel is adhered to the second hydrogel via the bridging polymer; wherein an adhesion between the first and second hydrogels is greater than or equal to 150 J/m² approximately about 5 seconds after contacting the first hydrogel and the dried bridging polymer film with the second portion of the hydrogel.

In an eighteenth embodiment, the present disclosure provides a method of applying the tough adhesive of the twelfth, thirteenth, fourteenth, fifteenth, or sixteenth embodiment, wherein the method comprises the step of compressing the dried bridging polymer either between a first and a second portion of the hydrogel or directly onto the hydrogel.

In a nineteenth embodiment, the present disclosure provides a method of applying the tough adhesive of the seventeenth embodiment, wherein the method comprises the step of compressing the dried bridging polymer between the first and second hydrogels.

In a twentieth embodiment, for the method the eighteenth or nineteenth embodiment, the hydrogel and the dried bridging polymer are compressed for up to about 5 seconds, or the hydrogel and the dried bridging polymer are compressed for up to about 1 minute, or the hydrogel and the dried bridging polymer are compressed for up to about 10 minutes.

In a twenty-first embodiment, for the tough adhesive of the twelfth, thirteenth, fourteenth, fifteenth, sixteenth, or seventeenth embodiment, an adhesion between said tough adhesive and an elastomer is greater than or equal to 150 J/m² approximately about 5 seconds after contacting said tough adhesive with said elastomer.

In a twenty-second embodiment, for the tough adhesive of twenty-first embodiment, the elastomer is a poly (acrylate) elastomer.

In another embodiment, the chitosan film is infused with a drug. In some embodiments the drug is 5-fluorouracil (5-FU).

In one embodiment, the chitosan film infused with a drug is adhered to a hydrogel as described in the embodiments above. In another embodiment, the chitosan film infused with a drug is placed in between a first and a second hydrogel. In another embodiment, the chitosan film infused with a drug is placed in-between a first and a second elastomer. In another embodiment, the chitosan film infused with a drug is placed in-between a hydrogel and an elastomer.

### BRIEF DESCRIPTION OF THE FIGURES

FIG. 1-1a—Schematic of a tough double network hydrogel matrix adhered to tissue surfaces via a bridging polymer (chitosan) that is proposed to yield topological entanglement with the hydrogel and underlying tissue. The double network is composed of covalently crosslinked polyacrylamide (pAAm) and calcium crosslinked alginate.

FIG. 1-1b—The effect of time on chitosan-only adhesion. Data shown as mean ± s.d. as evaluated by a one-way ANOVA with post hoc t-tests with Bonferroni corrections (n=4-6 samples/group).

FIG. 1-1c—Adhesion energy to skin in the presence and absence of EDC/sNHS coupling agents. Data shown as mean ± s.d. as evaluated by a Student's t-test.

FIG. 1-1d—The effect of tissue type on chitosan-only mediated adhesion. Data shown as mean ± s.d. as evaluated by one-way ANOVA with post hoc t-tests with Bonferroni corrections (n=6-8 samples/group). Adhesion tests were performed using tough gels prepared with alginate D and pAAm, and chitosan A as the bridging polymer.

FIG. 1-1e—Images showing tough adhesion of the tough adhesive to diverse tissue surfaces (skin, tendon, heart).

FIG. 1-2a—The effect of blood contaminating skin on the adhesion energy was evaluated. Data shown as mean ± s.d. as evaluated by Student's T-tests (n=6 samples/group).

FIG. 1-2b—The effect of incubation in DMEM after placement on the resulting adhesion energy was non-significant. Data shown as mean ± s.d. as evaluated by a Student T-tests (n=6-9 samples/group). Adhesion tests were performed using tough gels prepared with alginate D and pAAm, and chitosan A as the bridging polymer.

FIG. 1-3a—Proposed mechanism of topological entanglement by interchain interactions through H-bonds.

FIG. 1-3b—The effect of skin pH on the adhesion energy to skin tissue. Data shown as mean ± s.d., as evaluated by a one-way ANOVA with post hoc T-tests with Bonferroni corrections (n=4group).

FIG. 1-3c—The effect of chitosan concentration on the adhesion energy to skin tissue. Data shown as mean ± s.d., as evaluated by a one-way ANOVA with post hoc T-tests with Bonferroni corrections (n=4-5/group).

FIG. 1-3d—The effect of chitosan MW/purity on the adhesion energy. Data shown as mean ± s.d., as evaluated by a Student's t-test (n=4-7/group). Chitosan D was used as the high MW bridging polymer.

FIG. 1-3e—The effect of chitosan degree of deacetylation on the adhesion energy. Data shown as mean ± s.d., as evaluated by a Student's t-test (n=4-7/group). Chitosan A was used as the lower degree of deacetylation (DDA).

FIG. 1-3f—The effect of chitosan MW/purity and concentration on chitosan viscosity. Data shown as mean ± s.d., as evaluated by a two-way ANOVA with post hoc T-tests with Bonferroni corrections (n=10 samples/group). Adhesion tests were performed using tough gels prepared with alginate D and pAAm, and chitosan A as the bridging polymer. Chitosan D was used as the high MW bridging polymer.

FIG. 1-4a—The effect of alginate molecular weight on hydrogel toughness evaluated using both ultrapure (UP) and non-ultrapure (Non-UP) polymers. For the high MW alginates A and C were used, while alginates C and F were used as the low MW alginates. Data shown as mean ± s.d., as evaluated by a one-way ANOVA with post hoc t-tests with Bonferroni corrections (n=4-6 samples/group). This adhesion test was performed using tough gels with alginates A or C as the UP materials, alginates E and F as the Non-UP, and chitosan A.

FIG. 1-4b—The effect of alginate molecular weight on hydrogel modulus evaluated using both ultrapure (UP) and non-ultrapure (Non-UP) polymers. For the high MW alginates A and C were used, while alginates C and F were used as the low MW alginates. Data shown as mean ± s.d., as evaluated by a one-way ANOVA with post hoc t-tests with Bonferroni corrections (n=4-6 samples/group). This adhesion test was performed using tough gels with alginates A or C as the UP materials, alginates E and F as the Non-UP, and chitosan A.

FIG. 1-4c—The effect of alginate molecular weight on hydrogel the adhesion energy evaluated using both ultrapure (UP) and non-ultrapure (Non-UP) polymers. For the high MW alginates A and C were used, while alginates C and F were used as the low MW alginates. Data shown as mean ± s.d., as evaluated by a one-way ANOVA with post hoc t-tests with Bonferroni corrections (n=4-6 samples/group). This adhesion test was performed using tough gels with alginates A or C as the UP materials, alginates E and F as the Non-UP, and chitosan A.

FIG. 1-4d—Graph showing that the adhesion energy was strongly correlated with alginate molecular weight (R²=0.7166, P<0.001).

FIG. 1-5a—Schematic of proposed mechanism for rapid adhesion. TG: tough gel.

FIG. 1-5b—The effect of time on tough hydrogel dehydration, rehydration, and swelling. “Regular” indicates swelling behavior of gels as prepared. “TG Films” indicates swelling behavior of dehydrated gels. TG: tough gel.

FIG. 1-5c—The effect of tough hydrogel dehydration on the adhesion energy and shear strength to porcine skin after 1-minute of application. Data shown as mean ± s.d., as evaluated by a Student's t-test (n=3-4 samples/group). Adhesion tests were performed using tough gels prepared with alginate D and pAAm, and chitosan A as the bridging polymer.

FIG. 1-5d—The effect of tough hydrogel hydration and time on chitosan interpenetration at 1 minute. Chitosan was labeled with FITC, and the skin was visualized with polarized light. Scale bar=100μm. Data shown as mean ± s.d., as evaluated by a two-way ANVOA with post hoc t-tests with Bonferroni corrections (n=3-4 samples/group). TG: tough gel; C: chitosan; S: skin.

FIG. 1-S1a—Gel adhesion resulted in steep peak forces during peel testing that corresponded to bands of cohesive failure in the hydrogels.

FIG. 1-S1b—Image showing the bands of cohesive failure in the hydrogels.

FIG. 1-S2a—Effect of blood exposure on the adhesion energy with and without EDC/sNHS mixed into the chitosan bridging polymer.

FIG. 1-S2b—Adhesion energy after 24 h of incubation in DMEM with and without EDC/sNHS mixed into the chitosan bridging polymer. P-values reported after Student's t-test. Data shown as mean ± s.d. with N=5-6 samples/group.

FIG. 1-S3a—Confocal images of FITC-labeled chitosan (green) between skin (white, polarized light) and gels (dark). Blue indicates cell nuclei (DAPI). Scale bar=100μm. The images show the effect of chitosan concentration on penetration into skin and the total chitosan interfacial thickness.

FIG. 1-S3b—The effect of chitosan concentration on penetration into skin. P-values reported after Student's t-test. Data shown as mean ± s.d. with N=5 samples/group. Adhesion tests were performed using tough gels prepared with alginate D and pAAm, and fluorescently labelled chitosan A as the bridging polymer.

FIG. 1-S3c—The effect of chitosan concentration on the total chitosan interfacial thickness. P-values reported after Student's t-test. Data shown as mean ± s.d. with N=5 samples/group. Adhesion tests were performed using tough gels prepared with alginate D and pAAm, and fluorescently labelled chitosan A as the bridging polymer.

FIG. 1-S4a—Characterization of the UP (med MW, 95% DDA) vs Non-UP (high MW. 76% DDA) chitosans. FTIR of Ultrapure Med MW Chitosan (chitosan A) and Non-UP High MW Chitosan (chitosan D).

FIG. 1-S4b—Characterization of the UP (med MW, 95% DDA) vs Non-UP (high MW, 76% DDA) chitosans. Thermogravimetric analysis (TGA) demonstrating thermal decomposition of Ultrapure Med MW Chitosan (chitosan A) and Non-UP High MW Chitosan (chitosan D).

FIG. 1-S5—Image showing the tissue adhesion energy with different chitosans. Adhesion tests were performed using tough gels prepared with alginate D and pAAm.

FIG. 1-S6a—Plots and Pearson's correlations between between toughness and average molecular weight of the alginate chains. (R²), P<0.0001.

FIG. 1-S6b—Plots and Pearson's correlations between between stretch and average molecular weight of the alginate chains. (R²), P<0.0001.

FIG. 1-S6c—Plots and Pearson's correlations between between maximum stretch and average molecular weight of the alginate chains. (R²), P<0.0001.

FIG. 1-S6d—Plots and Pearson's correlations between between modulus and average molecular weight of the alginate chains. (R²), P<0.0001.

FIG. 1-S7a—Plots and Pearson's correlations between adhesion energy and toughness of gels fabricated with different alginates. (R²), P<0.0001.

FIG. 1-S7b—Plots and Pearson's correlations between toughness and modulus of gels fabricated with different alginates. (R²), P<0.0001.

FIG. 1-S7c—Plots and Pearson's correlations between adhesion energy and modulus of gels fabricated with different alginates. (R²), P<0.0001.

FIG. 1-S8a—Schematic of the G-block content in the alginates.

FIG. 1-S8b—Effect of the G-block content on toughness. Data shown as mean ± s.d., with N=4 samples/group. Gels were crosslinked with 14 mM Ca²⁺. P-values are shown after Student's t-tests. Tests performed using tough gels with alginate B or B2 as polymer chains with same MW but different G-block content. All tests were performed using chitosan A.

FIG. 1-S8c—Effect of the G-block content on stretch. Data shown as mean ± s.d., with N=4 samples/group. Gels were crosslinked with 14 mM Ca²⁺. P-values are shown after Student's t-tests. Tests performed using tough gels with alginate B or B2 as polymer chains with same MW but different G-block content. All tests were performed using chitosan A.

FIG. 1-S8d—Effect of the G-block content on maximum stretch. Data shown as mean ± s.d., with N=4 samples/group. Gels were crosslinked with 14 mM Ca²⁺. P-values are shown after Student's t-tests. Tests performed using tough gels with alginate B or B2 as polymer chains with same MW but different G-block content. All tests were performed using chitosan A.

FIG. 1-S8e—Effect of the G-block content on modulus. Data shown as mean ± s.d., with N=4 samples/group. Gels were crosslinked with 14 mM Ca²⁺. P-values are shown after Student's t-tests. Tests performed using tough gels with alginate B or B2 as polymer chains with same MW but different G-block content. All tests were performed using chitosan A.

FIG. 1-S8f—Effect of the G-block content on adhesion energy. Data shown as mean ± s.d., with N=4 samples/group. Gels were crosslinked with 14 mM Ca²⁺. P-values are shown after Student's t-tests. Tests performed using tough gels with alginate B or B2 as polymer chains with same MW but different G-block content. All tests were performed using chitosan A.

FIG. 1-S9—Effect of combining high and low molecular weight alginates on the adhesion energy. Adhesion energy using double network hydrogels with different alginates. Unmanipulated LF20/40 alginate (alginate E), LF20/40 irradiated at 5 mRad prior to gel fabrication (alginate F), and a 1:1 combination of the high molecular weight LF20/40 and low molecular weight irradiated alginate were used to fabricate gels. Data shown as mean ± s.d., with N=4 samples/group. P-values are shown after one-way ANOVA with post hoc t-tests with Bonferroni corrections. Adhesion tests performed using chitosan A as the bridging polymer.

FIG. 1-S10a—Schematic showing how gels were implanted subcutaneously in mice and examined longitudinally using high frequency ultrasound (HFUS) for two weeks. Sagittal B-mode images were acquired to determine the gel thickness.

FIG. 1-S10b—Sagittal B-mode images acquired to determine the gel thickness. Yellow dotted lines show the sides of the gels used to calculated the thickness.

FIG. 1-S10c—Graphs showing the thickness of gels, implanted in hydrated (left column) and dehydrated state (right column), over time in vivo. Data shown as mean ± s.d., N=3-5 gels/group. P-values shown as analyzed by a two-way ANOVA with post hoc tests with Bonferroni corrections.

FIG. 1-S11a—The effect of film dehydration on the failure stress. Data shown as mean ± s.d., with N=4-5 samples/group. P-value shown after Student's t-test. All gels were prepared with alginate D and pAAm.

FIG. 1-S11a—The effect of film dehydration on the linear modulus. Data shown as mean ± s.d., with N=4-5 samples/group. P-value shown after Student's t-test. All gels were prepared with alginate D and pAAm.

FIG. 2-S1a—The effect of film thickness on the maximum stress. Data shown as mean ± s.d. (n=4 samples/group), as evaluated by a Student's t-test.

FIG. 2-S1b—The effect of film thickness on the modulus. Data shown as mean ± s.d. (n=4 samples/group), as evaluated by a Student's t-test.

FIG. 2-S1c—The effect of film thickness on the maximum stretch. Data shown as mean ± s.d. (n=4 samples/group), as evaluated by a Student's t-test.

FIG. 2-S1d—The effect of film thickness on the adhesion energy. Data shown as mean ± s.d. (n=4 samples/group), as evaluated by a Student's t-test.

FIG. 2-1a—Schematic highlighting differences between liquid and film-based adhesion generation.

FIG. 2-1b—EDS-based elemental mapping of the interfaces using liquid chitosan (C) and chitosan films (CFs) applied between alginate-polyacrylamide tough gels (TG). In both cases, the bridging layer was rich in Cl⁻, a surrogate measure for chitosan (chitosan-HCl); Scale bar: 100μm.

FIG. 2-1c—Images showing how after application of the CF between two TGs, adhesion occurred instantly.

FIG. 2-1d—Images showing robust adhesion linking two gels is demonstrated as gels maintained apposition despite high tension. Yellow lines indicate borders of the CF after attachment to the TG.

FIG. 2-2a—The effect of bridging film on the adhesion energy between Alg-PAAM gels. Data shown as mean ± s.d (n=4 gels/group), as evaluated by a one-way ANOVA with post hoc t-tests with Bonferroni corrections.

FIG. 2-2b—Application of CFs to TGs led to wrinkling immediately after attachment macroscopically (top) and under confocal imaging (bottom). Red indicates TG and green indicates CF.

FIG. 2-2c—The effect of incubation time in DMEM at 37° C. on the adhesion energy between Alg-PAAm gels. Data shown as mean ± s.d (n=4-7 gels/group), as evaluated by a one-way ANOVA with post hoc t-tests with Bonferroni corrections.

FIG. 2-2d—Schematic of instant elastomer attachment (VHB, 3M) with CFs.

FIG. 2-2e—Image showing strong adhesion (up to 4000 J/m²) observed with CF to VHB elastomer.

FIG. 2-2f—Stability of TG-CF attached to VHB elastomer after 0 and 24 hours of incubation in DMEM at 37° C. Dashed lines indicate comparison to the benzophenone strategy for elastomer attachment as prepared and fully swollen TGs (18.1).

FIG. 2-3a—Schematic showing the effect of electrostatics on adhesion.

FIG. 2-3b—The effect of electrostatics on adhesion. Data shown as mean ± s.d. (n=3-4 gels/group), as evaluated by a Student's t-test.

FIG. 2-3c—Schematic showing the effect of TG pH on adhesion.

FIG. 2-3d—The effect of TG pH on adhesion. Data shown as mean ± s.d. (n=3-4 gels/group), as evaluated by a Student's t-test.

FIG. 2-3e—Schematic showing the effect of prior CF deprotonation on adhesion.

FIG. 2-3f—The effect of prior CF deprotonation on adhesion. Data shown as mean ± s.d. (n=2-6 gels/group), as evaluated by a Student's t-test.

FIG. 2-3g—Schematic showing the effect of dangling chain ends and surface entanglement on Alg-PAAm gel adhesion.

FIG. 2-3h—The effect of dangling chain ends and surface entanglement on Alg-PAAm gel adhesion. Data shown as mean ± s.d. (n=3-6 gels/group), as evaluated by a Student's t-test.

FIG. 2-4a—Schematic showing how the chitosan films (CF) function both to instantly self-adhere with (1) tough gels (TG) or (2) tough adhesives (TA) for several indications including: topical (skin) and internal (bowel, tendon, nerve, vessels). TG: Alg-PAAm only. TA: Alg-PAAm + liquid chitosan.

FIG. 2-4b—Schematic showing the application of the self-adhering TG around a finger.

FIG. 2-4c—Graph showing how the TG resulted in local skin cooling in contrast to TenderCare®. Data shown as individual points before and after application (n=7 hands/group), as evaluated by a two-way ANOVA for time and treatment.

FIG. 2-4d—Image showing how the TG+CF is easily wrapped around bowel tissue to provide self-adhesion but anti-adhesive properties to underlying tissue and surrounding organs.

FIG. 2-4e—Image showing how the TG+CF is easily wrapped around tendon tissue to provide self-adhesion but anti-adhesive properties to underlying tissue and surrounding organs.

FIG. 2-4f—Image showing how the TG+CF is easily wrapped around peripheral nervous tissue to provide self-adhesion but anti-adhesive properties to underlying tissue and surrounding organs.

FIG. 2-4g—High frequency ultrasound imaging confirmed apposition of the gels to tendon and nerve tissue. Scale bar=1 mm.

FIG. 2-4h—High frequency ultrasound imaging confirmed apposition of the gels to tendon and nerve tissue. Scale bar=1 mm.

FIG. 2-4i—Image showing the application of the CF over a tough gel is used as an aortic sealant to increase its strength (C is liquid chitosan).

FIG. 2-4j—Image showing how the CF is easily applied over the surface of the TG after adhesion to porcine aorta and withstood cyclic loading.

FIG. 2-4k—The effect of CF thickness on maximum burst pressures. Data shown as mean ± s.d. (n=3 samples/group), as evaluated by a one-way ANOVA with post hoc t-tests with Bonferroni corrections.

FIG. 2-S2a—Image showing that the thinner chitosan films displayed more noticeable wrinkling on the surface of the TG.

FIG. 2-S2b—Confocal microscopy showing the interface between two tough gels TG adhered with a CF. Scale bar: 100μm. Red indicates nile-blue labeled TG (top and bottom sections) and green indicates FITC-labeled CF (middle section).

FIG. 2-S3—Confocal imaging showing the effect of time on the thickness of the chitosan films. Green indicates a FITC labeled CF. Scale bar=100μm.

FIG. 2-S4a—Confocal imagery showing residual PAAM observed on the bridging surface of the gel after peeling was completed. Red indicates nile blue fluorescent labeling of the TG and green indicates FITC labeled CF.

FIG. 2-S4b—Surface defects on the bridging surface gel after peeling. Red indicates nile blue fluorescent labeling of the TG and green indicates FITC labeled CF.

FIG. 2-S5a—The effect of varying polymers and buffers on the zeta potential. Data shown as mean +/−SD (n=3-6 samples/group).

FIG. 2-S5b—The effect of buffer selection on adhesion of polyacrylamide hydrogels. Data shown as mean +/−s.d. (n=3 samples/group), as evaluated by a Student's t-test.

FIG. 3-1—Cumulative 5-FU release over time for the single layer hydrogel design in comparison to the “sandwich” design.

FIG. 3-2—Cumulative 5-FU release over time for the different “sandwich” designs.

FIG. 3-3—Cumulative 5-FU release over time for the hydrogel “sandwich” design at different saline pH values.

### DETAILED DESCRIPTION

As used herein “surface” has the general meaning in the art, “the exterior or upper boundary of an object or body” (see Merriam-Webster dictionary). In some embodiments, the surface is a tissue. As used herein, the term “tissue” has the general meaning of the art. Tissue can refer to an organ, muscle, skin, or other group of cells which function together as a unit. In some embodiments the surface is an elastomer. As used herein, “elastomer” is a polymer which typically has elastic properties.

As used herein the term “ultra-pure” refers to a high purity which is over 60%, 70%, 80%, 90% or more. In some embodiments, “ultra-pure” refers to low levels of residual endotoxin, such as below 100 EU/g.

In some embodiments, the tough adhesive (TA, also referred to as a tissue adhesive) herein) includes a hydrogel that can be selectively activated with a bridging polymer. Without wishing to be bound by theory, it is believed that the surface of alginate-polyacrylamide hydrogels (e.g., an alginate-based hydrogel) is activated by the bridging polymer (e.g., chitosan). In particular, the bridging polymer is proposed to act as a stimuli-responsive polymer by forming strong intermolecular H-bonds upon a change in pH. This property allows the chains to diffuse and form an internal network between two permeable adherends, in this case, the hydrogel and tissue or hydrogel and hydrogel.

As used herein, the term “contacting” (e.g., contacting a surface) is intended to include any form of interaction (e.g., direct or indirect interaction) of a hydrogel and a surface (e.g., a tissue or a device). Contacting a surface with a composition may be performed either in vivo or in vitro. In certain embodiments, the surface is contacted with the tough adhesive in vitro and subsequently transferred into a surface in an ex vivo method of administration. Contacting the surface with the tough adhesive in vivo may be done, for example, by injecting the tough adhesive into the surface, or by injecting the tough adhesive into or around the surface.

In some embodiments, the hydrogel used in the tough adhesive of the invention is an interpenetrating network (IPN) hydrogel. As used herein, an IPN is a polymer comprising two or more networks (e.g., the first polymer network and the second polymer network) which are at least partially interlaced on a molecular scale but not covalently bonded to each other and cannot be separated unless chemical bonds are broken. IPN hydrogels are made by combining covalently crosslinked and ionically crosslinked polymer networks. Alternatively, the first polymer network and the second polymer network are covalently coupled.

In particular, the first polymer network comprises covalent crosslinks and includes a polymer selected from the group consisting of polyacrylamide (PAAM). poly(hydroxyethylmethacrylate) (PHEMA), poly(vinyl alcohol) (PVA), polyethylene glycol (PEG), polyphosphazene, collagen, gelatin, poly(acrylate), poly(methacrylate), poly(methacrylamide), poly(acrylic acid), poly(N-isopropylacrylamide) (PNIPAM), poly(N,N-dimentylacrylamide), poly(allylamine) and copolymers thereof. In a particular embodiment, the first polymer network is polyethylene glycol (PEG). In some embodiments, the first polymer network is polyacrylamide (PAAM).

The second polymer network includes ionic crosslinks and is a polymer selected from the group consisting of alginate (alginic acid or align), pectate (pectinic acid or polygalacturonic acid), carboxymethyl cellulose (CMC or cellulose gum), hyaluronate (hyaluronic acid or hyaluronan), chitosan, κ-carrageenan, ι-carrageenan and λ-carrageenan, wherein the alginate, carboxymethyl cellulose, hyaluronate, chitosan, κ-carrageenan, ι-carrageenan and λ-carrageenan are each optionally oxidized, wherein the alginate, hyaluronate, chitosan, κ-carrageenan, ι-carrageenan and λ-carrageenan optionally include one or more groups selected from the group consisting of methacrylate, acrylate. acrylamide, methacrylamide, thiol, hydrazine, tetrazine, norbornene, transcyclooctene and cyclooctyne.

In a particular embodiment, the second polymer network is alginate, which is comprised of (1-4)-linked b-D-mannuronic acid (M) and a-L-guluronic acid (G) monomers that vary in amount and sequential distribution along the polymer chain. Alginate is also considered a block copolymer, composed of sequential M units (M blocks), regions of sequential G units (G blocks), and regions of alternating M and G units (M-G blocks) that provide the molecule with its unique properties. Alginates have the ability to bind divalent cations such as Ca⁺² between the G blocks of adjacent alginate chains, creating ionic interchain bridges between flexible regions of M blocks.

In some embodiments, the alginate is a mixture of a high molecular weight alginate and a low molecular weight alginate. For example. the ratio of the high molecular weight alginate to the low molecular weight alginate is about 5:1 to about 1:5; about 4:1 to about 1:4; about 3:1 to about 1:3; about 2:1 to about 1:2; or about 1:1. In other embodiments the alginate is mostly or exclusively high molecular weight alginate, and in other embodiments the alginate is mostly or exclusively low molecular weight alginate. The high molecular weight alginate has a molecular weight from about 100 kDa to about 300 kDa, from about 150 kDa to about 250 kDa, or is about 200 kDa. In some embodiments the high molecular weight alginate has a molecular weight of about 100 kDa or more, and in other embodiments of about 200 kDa or more. The low molecular weight alginate has a molecular weight from about 1 kDa to about 100 kDa. from about 5 kDa to about 50 kDa, from about 10 kDa to about 30 kDa, or is about 20 kDa.

In some embodiments, the G-content of the alginate is ≥60. In other embodiments the G-content is 65-75. And in other embodiments, the G-content of the alginate is ≤50.

In some embodiments the hydrogel comprises only a single polymer network. In some embodiments the single polymer network comprises the covalently cross-linked polymers disclosed above and in other embodiments the single polymer network comprises the ionically cross-linked polymers disclosed above.

The hydrogels of the invention are highly absorbent and comprise about 30% to about 98% water (e.g., about 40%, about, about 50%, about 60%, about 70%, about 80%, about 90%, about 95%, about 98%, about 40 to about 98%, about 50 to about 98%, about 60 to about 98%, about 70 to about 98%, about 80 to about 98%, about 90 to about 98%, or about 95 to about 98% water) and possess a degree of flexibility similar to natural tissue, due to their significant water content. In particular, the hydrogels of the present invention can be stretched up to 20 times their initial length, e.g., the hydrogels of present invention can be stretched from 2 to 20 times their initial length, 5 to 20 times their initial length, 10 to 20 times their initial length. from 15 to 20 times their initial length, from 2 to 10 times their initial length, from 10 to 15 times their initial length, and from 5 to 15 times their initial length without cracking or tearing.

The tough adhesive includes a bridging polymer which is a primary amine polymer. The bridging polymer forms covalent bonds with both the hydrogel and a surface (e.g., a tissue or an elastomer), bridging the two. The bridging polymer is separate and distinct from the hydrogel. The primary amine polymer bears positively charged primary amine groups under physiological conditions. In some embodiments, the primary amine polymer can be absorbed to a surface (e.g., a tissue, a cell, an elastomer, or a device) via electrostatic interactions, and provide primary amine groups to bind covalently with both carboxylic acid groups in the hydrogel and on the surface. If the surface is permeable, the primary amine polymer can also penetrate into the surface, forming physical entanglements, and then chemically anchor the hydrogel.

As used herein, the primary amine polymer includes at least one primary amine per monomer unit. In some embodiments, the primary amine polymer is selected from the group consisting of chitosan, gelatin, collagen, polyallylamine, polylysine, and polyethylenimine. In some embodiments, the primary amine polymer is selected from the group consisting of chitosan, gelatin, collagen, polyallylamine, polylysine, polyethylenimine, poly(amino styrene) (PAS), poly(acrylic acid) (PAAc), and carboxymethyl chitosan (CMC). In some embodiments, the primary amine polymer is selected from chitosan, polyethylenamine (PEI), polyallylamine (PAA), and N,O-carboxymethyl chitosan (CMC). In some embodiments, the primary amine polymer is a proteoglycan (e.g. chondroitin sulfate or heparin sulfate). In particular, polyallylamine (PolyNH₂ or PAA) is represented by the following structural formula:

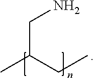

In particular, chitosan is represented by the following structural formula:

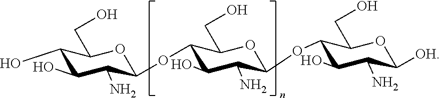

In particular, polyethylenimine (PEI) is represented by the following structural formula:

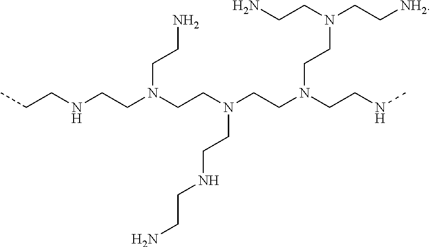

In particular, polylysine is represented by the following structural formula:

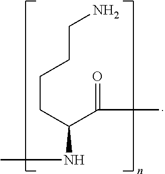

Collagen and/or gelatin include approximately ˜10% amino acid with primary amine (e.g., Arg, Lysine).

In some embodiments, the bridging polymer is a chitosan polymer. In some embodiments the chitosan polymer is a mixture of a high molecular weight chitosan and a low molecular weight chitosan. For example, the ratio of the high molecular weight chitosan to the low molecular weight chitosan is about 5:1 to about 1:5; about 4:1 to about 1:4; about 3:1 to about 1:3; about 2:1 to about 1:2; or about 1:1. In other embodiments the chitosan is mostly or exclusively high molecular weight chitosan, and in other embodiments the chitosan is mostly or exclusively low molecular weight chitosan. The high molecular weight chitosan has a molecular weight from about 200 kDa to about 500 kDa, from about 300 kDa to about 500 kDa, from about 400 kDa to about 500 kDa, or is about 500 kDa. The low molecular weight chitosan has a molecular weight from about 1 kDa to about 200 kDa, from about 100 kDa to about 190 kDa, from about 100 kDa to about 180 kDa, or is about 165 kDa.

In some embodiments, the concentration of chitosan is about 1% wt/volume to about 6% wt/volume. In some embodiments the concentration of chitosan is about 1% wt/volume to about 4% wt/volume, or about 1.5% wt/volume to about 3.5% wt/volume, or is about 2% wt/volume. In some embodiments the higher concentration of chitosan leads to a higher viscosity liquid. In some embodiments, the viscosity is between 0.1 and 600 Pa·s. In some embodiments the viscosity is about 0.2 Pa·s to about 4.0 Pa·s, or about 0.1 Pa·s to about 0.4 Pa·s, or about 1.0 to about 4.0 Pa·s, or about 300 Pa·s to about 1000 Pa·s.

In some embodiments the chitosan is ultra-pure (“UP”) chitosan and in other embodiments the chitosan is non-pure (“NP”).

In some embodiments the degree of deacetylation of the chitosan is greater than 60%. In some embodiments the degree of deacetylation is about 60% to about 100%, is about 60% yo about 80%, is about 80% to about 100%, or is about 90% to about 100%.

In some embodiments the first polymer network, the second polymer network, and the bridging polymer are all different materials.

As used herein, the term “dried” or “dehydrated” (or the process of “drying” or “dehydrating”) involves removing a substantial amount of residual water in a polymer. In some embodiments more than 10% of the water is removed, in others more than 20%, in others more than 30%, in others more than 40%, in others more than 50%, in others more than 60%, in others more than 70%. in others more than 80%, in others more than 90%, or in others more than 99%. The drying process can involve any method commonly known in the art, such as air drying, oven drying, flame drying, reduced pressure drying, or combinations thereof. In some embodiments drying takes places at room temperature, in other embodiments drying takes place with heating. In some embodiments where heating is used, the object is heated to up to 50° C. up to 100° C., or up to 150° C.

In some embodiments, the pH of the surface (e.g. a tissue or an elastomer) is about 0to about 14. In some embodiments, the pH is about 2 to about 12, or about 4 to about 12, or about 4 to about 9, or about 6 to about 8, or about 8 to about 12, or about 8 to about 9.

In some embodiments the tough adhesive is adhered to an elastomer. In some embodiments, the elastomer is made up of one or more polymers selected from the group consisting of polyacrylamide (PAAM), poly(hydroxyethylmethacrylate) (PHEMA), poly(vinyl alcohol) (PVA), polyethylene glycol (PEG), polyphosphazene, collagen, gelatin, poly(acrylate), poly(methacrylate), poly(methacrylamide), poly(acrylic acid), poly(N-isopropylacrylamide) (PNIPAM), poly(N,N-dimentylacrylamide), poly(allylamine) and copolymers thereof. In a particular embodiment. the elastomer is a poly(acrylate) polymer. In some embodiments, the elastomer is a commercial elastomer, for example, the “VHB” polymer by 3M. In some embodiments the VHB polymer comprises carbonyl and/or alcohol groups.

In some embodiments, the adhesion energy (or adhesion strength) is measured between the tough adhesive and/or the hydrogel and a surface (e.g., a tissue or an elastomer). In some embodiments, the adhesion energy is measured between the tough adhesive and the elastomer. In some embodiments, the adhesion energy is measured between the tough adhesive and a tissue. In some embodiments, the adhesion energy is measured between one or more hydrogels and the bridging polymer. In some embodiments, the adhesion energy is measured between one or more hydrogels and a dried bridging polymer film.

In some embodiments, the adhesion energy is greater than or equal to 150 J/m² after about 5 seconds of contact, or greater than or equal to 300 J/m² after about 5 seconds of contact, or greater than or equal to 400 J/m² after about 5 seconds of contact, or greater than or equal to 500 J/m² after about 5 seconds of contact, or greater than or equal to 800 J/m² after about 5 seconds of contact, or greater than or equal to 1000 J/m² after about 5 seconds of contact, or greater than or equal to 1500 J/m² after about 5 seconds of contact, or greater than or equal to 2000 J/m² after about 5 seconds of contact, or greater than or equal to 2500 J/m² after about 5 seconds of contact, or greater than or equal to 4000 J/m² after about 5 seconds of contact.

In some embodiments, the adhesion energy is greater than or equal to 150 J/m² after about 1 minute of contact, or greater than or equal to 300 J/m² after about 1 minute of contact, or greater than or equal to 400 J/m² after about 1 minute of contact, or greater than or equal to 500 J/m² after about 1 minute of contact, or greater than or equal to 800 J/m² after about 1 minute of contact, or greater than or equal to 1000 J/m² after about 1 minute of contact, or greater than or equal to 1500 J/m² after about 1 minute of contact, or greater than or equal to 2000 J/m² after about 1 minute of contact, or greater than or equal to 2500 J/m² after about 1 minute of contact, or greater than or equal to 4000 J/m² after about 1 minute of contact.

In some embodiments, the adhesion energy is greater than or equal to 150 J/m² after about 3 minutes of contact, or greater than or equal to 300 J/m² after about 3 minutes of contact, or greater than or equal to 400 J/m² after about 3 minutes of contact, or greater than or equal to 500 J/m² after about 3 minutes of contact, or greater than or equal to 800 J/m² after about 3 minutes of contact, or greater than or equal to 1000 J/m² after about 3 minutes of contact, or greater than or equal to 1500 J/m² after about 3 minutes of contact, or greater than or equal to 2000 J/m² after about 3 minutes of contact, or greater than or equal to 2500 J/m² after about 3 minutes of contact, or greater than or equal to 4000 J/m² after about 3 minutes of contact.

In some embodiments, the adhesion energy is greater than or equal to 150 J/m² after about 5 minutes of contact, or greater than or equal to 300 J/m² after about 5 minutes of contact, or greater than or equal to 400 J/m² after about 5 minutes of contact, or greater than or equal to 500 J/m² after about 5 minutes of contact, or greater than or equal to 800 J/m² after about 5 minutes of contact, or greater than or equal to 1000 J/m² after about 5 minutes of contact, or greater than or equal to 1500 J/m² after about 5 minutes of contact, or greater than or equal to 2000 J/m² after about 5 minutes of contact, or greater than or equal to 2500 J/m² after about 5 minutes of contact, or greater than or equal to 4000 J/m² after about 5 minutes of contact.

In some embodiments, the adhesion energy is greater than or equal to 150 J/m² after about 10 minutes of contact, or greater than or equal to 300 J/m² after about 10 minutes of contact, or greater than or equal to 400 J/m² after about 10 minutes of contact, or greater than or equal to 500 J/m² after about 10 minutes of contact, or greater than or equal to 800 J/m² after about 10 minutes of contact, or greater than or equal to 1000 J/m² after about 10 minutes of contact, or greater than or equal to 1500 J/m² after about 10 minutes of contact, or greater than or equal to 2000 J/m² after about 10 minutes of contact, or greater than or equal to 2500 J/m² after about 10 minutes of contact, or greater than or equal to 4000 J/m² after about 10 minutes of contact.

The present invention also provides a method of adhering a hydrogel to a surface, the method including the steps of a) adding a bridging polymer disclosed herein to said hydrogel; and b) compressing said hydrogel gel with said bridging polymer onto said surface. In certain embodiments, the surface is a tissue. The system can be applied to any tissue, including, but not limited to, heart tissue, skin tissue, blood vessel tissue, bowel tissue, liver tissue, kidney tissue, pancreatic tissue, lung tissue, trachea tissue, eye tissue, cartilage tissue, tendon tissue. Alternatively, the surface is a medical device. The system can be applied to any tissue, including, but not limited to, the group consisting of a defibrillator, a pacemaker, a stent, a catheter, a tissue implant, a screw, a pin, a plate, a rod, an artificial joint, a elastomer-based (e.g., PDMS, PTU) device, a hydrogel-based device (e.g., scaffolds for drug or cell delivery or sensors), and sensors for measuring, for example, temperature, pH, and local tissue strains.

The present invention also includes methods to encapsulate a medical device, or to coat a surface of a device. In particular, the hydrogel and the high density primary amine polymer are applied to the exterior surface of the hydrogel, and then the hydrogel is applied to the surface of the device. The high density primary amine polymer adhere the hydrogel to the surface of the device. Depending upon to desired outcome, the device can be completely encapsulated by the hydrogel or partially encapsulated, leaving some surface of the device exposed. Specifically, a “partially encapsulated” device refers to coating the device either on one surface of the device (e.g., the back, front or sides of the device) or on one portion of the device (e.g., the bottom half or the top half). In a particular embodiment, the high density primary amine polymer may be applied to multiple sites of the hydrogel so that the hydrogel can adhere to both the device and also another surface (e.g., a tissue).

The present invention also method to close a wound or injury and promote wound healing. In particular, the hydrogel and the high density primary amine polymer are applied to the exterior surface of the hydrogel, and then the hydrogel is applied to the location of the wound or injury. In a particular embodiment, the hydrogel is applied to the heart in order to repair a heart defect.

The present invention also includes methods of delivering a therapeutically active agent to a subject, the method including a) applying a solution comprising a high density primary amine polymer to a hydrogel; and b) placing the hydrogel on the surface; wherein the hydrogel comprises a first polymer network and a second polymer network, wherein the first polymer network comprises covalent crosslinks and the second polymer network comprises ionic crosslinks, and wherein at least one therapeutically active agent is encapsulated in, or attached to the surface of, the hydrogel and/or high density primary amine polymer, thereby delivering a therapeutically active agent to the subject.

The methods of the present invention include contacting the surface with a biocompatible adhesive. The surface can be contacted with the composition by any known routes in the art. As used herein, the term “delivery” refers to the placement of a composition into a subject by a method or route which results in at least partial localization of the composition at a desired site such that a desired effect is produced.

Exemplary modes of delivery include, but are not limited to, injection, insertion, implantation, or delivery within a scaffold that encapsulates the composition of the invention at the target surface, e.g., a tissue or organ. When the compositions of the invention are dissolved in a solution, they can be injected into the surface by a syringe.

The methods of the present invention are suitable for medical purposes, e.g., wound closure, delivery of a therapeutic agent, or attachment of a medical device, in a subject, wherein the subject is a mammal. In some embodiments, a mammal is a primate, e.g., a human or an animal. Usually the animal is a vertebrate such as a primate, rodent, domestic animal or game animal. Primates include chimpanzees, cynomologous monkeys, spider monkeys, and macaques, e.g., Rhesus. Rodents include mice, rats, woodchucks, ferrets, rabbits and hamsters. Domestic and game animals include cows, horses, pigs, deer, bison, buffalo, feline species, e.g., domestic cat, canine species, e.g., dog, fox, wolf, avian species, e.g., chicken, emu, ostrich, and fish, e.g., trout, catfish and salmon. In some embodiments, a subject is selected from the group consisting of a human, a dog, a pig, a cow, a rabbit, a horse, a cat, a mouse and a rat. In preferred embodiments, the subject is a human.

Exemplary modes of delivery include, but are not limited to, injection, insertion, implantation, or delivery within a scaffold that encapsulates the composition of the invention at the target tissue. In some embodiments, the composition is delivered to a natural or artificial cavity or chamber of a tooth of a subject by injection. When the compositions of the invention are dissolved in a solution, they can be injected into the tissue by a syringe.

In another aspect, the present invention provides a method of adhering a hydrogel to a surface (e.g., tissue or device), the method including the steps of a) applying a solution comprising a high density primary amine polymer to the hydrogel; and b) placing the hydrogel on the surface; wherein the hydrogel comprises a first polymer network and a second polymer network, wherein the first polymer network comprises covalent crosslinks and the second polymer network comprises ionic crosslinks.

In some embodiments, the chitosan film is infused with a drug or other molecule, such as 5-fluorouracil (5-FU). In some embodiments, the infused molecule is released over time in a solution. In some embodiments, the chitosan film, either alone or in combination with one or more hydrogel(s) and/or elastomer(s) are adhered to a tissue and the infused molecule is released over time around the tissue. In some embodiments the infused molecule is released at a pH of greater than approximately 3, greater than approximately 5, or greater than approximately 7. In other embodiments the infused molecule is released in a pH of approximately 2 to approximately 9, approximately 3 to approximately 7, or approximately 3 to approximately 5. In some embodiments the infused molecule is released at a pH of approximately 5.5 or a pH of approximately 3.5. In some embodiments the chitosan film infused with a molecule is placed between a first and second hydrogel, between a first and second elastomer, or between an elastomer and a hydrogel.

### Methods

### Tough Hydrogel Synthesis

Hydrogels were synthesized by mixing one syringe containing a 14 mL solution of 2.2% sodium alginate (summary of alginates in Table 1-S2) and 13.5% acrylamide (Sigma, A8887) in HBSS (Gibco), 50.4 μl of 2% N,N′-methylenebis(acrylamide) (Sigma, M7279), and 11.2 μL of TEMED (Sigma, T7024), with a second syringe containing 316.4 μl of 6.6% ammonium persulfate (Sigma, A9164), and 267.4 μl of 0.75M calcium sulfate dihydrate (Sigma, 31221). Alginates with different molecular weights (see Table 1-S2 for details) were obtained from NovaMatrix (MVG, LVG, LVM, VLVG) and FMC BioPolymer (LF10/60, LF20/40). Low molecular weight sodium alginate (LF20-40-5Mrad) was prepared by irradiating the high molecular weight sodium alginate (LF20/40) under γ-rays at a dose of 5 Mrad.^{[31]} The gel was cast into glass molds (110×15×1.7 mm³) sealed on both sides with glass and left to crosslink for 24 h. After 24 h, tough gel strips were removed from molds and stored in sealed plastic bags at 4° C. For hydrogels with lower pH, the same procedure was repeated, but with MES buffer. Alternatively, the hydrogel was dehydrated as discussed below, and re-hydrated with an HBSS buffer with a different pH.

To generate dehydrated films, hydrogels with LF10/60 alginate were placed over glass and left to dry at room temperature exposed to ambient air overnight. The obtained films were then transferred into sealed plastic bags to stop further dehydration.

TABLE 1-S2
Specifications of the alginates tested in this study.

| | Alginate Type | | | | | | |
|---|---|---|---|---|---|---|---|
| | A | B | — | C | D | E | F |
| Product # | MVG | LVG | LVM | VLVG | LF10/60 | LF20/40 | LF20/40-5mRad |
| Manufacturer | Nova | Nova | Nova | Nova | FMC | FMC | FMC |
| | Matrix | Matrix | Matrix | Matrix | BioPolymer | BioPolymer | BioPolymer, |
| | | | | | | | γ-Irradiated |
| MW (kDa) | 236 | 148 | — | 35 | 134 | 209 | 39 |
| PDI | 2.05 | 1.84 | — | 1.69 | 2.42 | 2.02 | 2.06 |
| G-Content | ≥60 | ≥60 | ≤50 | ≥60 | 65-75 | ≥60 | ≥60 |

*All values as reported by the manufacturer except for molecular weights (MW). MW was measured by GPC.

### Polyacrylamide Hydrogel Synthesis

A similar procedure was used, but in this case no alginate was added to the 13.5% acrylamide stock solution. The gels was cast into glass molds (110×15×1.7 mm³) sealed on both sides with glass and left to crosslink for 24 h. After 24 h, tough gel strips were removed from molds and stored in sealed plastic bags at 4° C.

### Generation of Polymeric Films

Polymer films (chitosan, PAS, pAAc, CMC) were generated by first dissolving polymers in water at 2% and 4% concentration and casting 10 mL of solution into molds on glass (6.5×9.5 cm). Ultrapure chitosan HCl (HMC, #54046), N,O-carboxymethyl chitosan (HMC, #44002), pAAC (Sigma, 323667), and PAS (Polysciences, #02823-1) were obtained. Samples were then placed in a 55C oven for 6 hours to dry. Dried samples were stored in sealed bags and desicant.

### Hydrogel Mechanical Properties

Pure shear tests were carried out to measure the matrix toughness. In brief, rectangular specimens (20×5×1.7 mm³) were tested in tension (Instron 3342, 10N load cell) at 100 mm/min. From the stress-stretch curves, the matrix maximum stretch, maximum stress, and fracture toughness were calculated^{[8]} using custom MATLAB code.

### Adhesive Application and Adhesion Energy Measurement

Chitosan (0.5-4% wt/vol) with or without coupling reagents (1-ethyl-3-(3-dimethylaminopropyl) carbodiimide (Sigma E6383) and sulfated N-hydroxy-succinimide) (Thermofisher, PG82071) (12 mg/mL) were quickly mixed in water by vortexing. A final concentration of 12 mg/ml EDC/sNHS was used in adhesion experiments with coupling reagents. A final pH of ˜5.5 was obtained in all chitosan solutions either by direct dissolution or by tuning with the addition of 0.1M HCl or 0.1M NaOH. This mixture was applied to the surface of the tough gel (50 μL/cm²) (0.5 mL total/sample) and compressed to tissue surfaces. Ultrapure Chitosan HCl (54046 and 54039) with different degrees of deacetylation were obtained from Heppe Medical Chitosan (HMC), Halle, Germany (see Table 1-S1 for details). High molecular weight chitosan (419419) was obtained from Sigma. A summary of chitosans used is provided in Table 1-S1. HBSS (Gibco) was used as the buffer in all cases unless stated otherwise.

TABLE 1-S1
Specifications of the chitosan tested in this study.

| Chitosan | Vendor/ | MW | Degree of | | |
|---|---|---|---|---|---|
| Type | Product | (kDa) | Deacetylation | Purity | Source |
| A | HMC | 170 | 95.4% | UP | Shrimp |
| | 54046 | | | | |
| B | HMC | 161 | 84.2% | UP | Shrimp |
| | 54039 | | | | |
| C | HMC | 465 | 96.4% | Non-Pure | Shrimp |
| | 95/2500 | | | | |
| D | Sigma | 495 | 76.0% | Non-Pure | Shrimp |
| | 419419 | | | | |
| E | CM95 | 202 | 98.0% | Non-Pure | Mushroom |

*All values as reported by the manufacturer except for molecular weights (MW). MW was measured by GPC.

Adhesion energy was measured with 180° peeling tests (Instron 3342) under uniaxial tension (100 mm/min). A tissue strip (˜75×11mm²) was placed over the gel and compression was applied for 10 mins unless stated otherwise. Applied compression strain was kept at ˜5.5% in all cases. The back of TA was also bonded to a rigid polyethylene terephthalate (PET) film with cyanoacrylate (Krazy Glue), in order to limit deformation to the crack tip, and thus all the work done by the machine would be equal to the energy dissipated at the crack tip. The free ends of TA and the substrate were attached to acrylic pieces, to which the machine grips were attached. A mechanical testing system (Instron 3342, 50N load cell) was used to apply unidirectional tension, while recording the force and the extension. The loading rate was kept constant at 100 mm/min. The adhesion energy was two times the peak value of the ratio of the force and width.^{[1]}

To test the adhesive in the presence of blood, the surface of tissue strips were fully covered with 300 μL of bovine blood per sample. The same procedure was then performed to compress the gel against the blood-covered tissue, and initiate adhesion. To study the effect of tissue pH on adhesion, porcine skin strips were equilibrated with buffers of different pH ranging from 4.0 to 8.5. First, MES (Sigma) buffer solutions with pH 4.0, 5.5, 7.0, and 8.5 were prepared by adding 0.1M NaOH and monitoring the solutions with a pH meter. Tissue strips (N=4 per group) were then placed in petri dishes filled with the corresponding buffering solutions and allowed to equilibrate for 20 minutes before testing. pH of the solutions was monitored with pH strips to make sure there was no pH change during the equilibration period. The skin samples were then removed from the buffers and tested following the same procedure as before.

### Interpenetration of the Chitosan Chains and Confocal Imaging

The skin-gel adhesive interface and chitosan diffusion profiles for both the hydrated and dehydrated tough gels were studied by using confocal imaging and fluorescently labeled chitosan (FITC Chitosan). FITC Chitosan was synthesized by reacting fluorescein isothiocyanate (Sigma, 1245460250) with chitosan. Briefly, 1 g of ultrapure chitosan HCl was dissolved in 100 mL of 0.1M acetic acid and 100 mg of FITC was dissolved in 100 mL of anhydrous methanol at 1.0 mg/mL in separate flasks. The FITC solution was then slowly added to the chitosan solution with continuous stirring. After ˜3 h, the reaction was quenched by slowly adding NaOH (0.5M) to increase the pH to ˜10, precipitating the fluorescently labelled chitosan. The solutions were then centrifuged, and dIH2O was added to the precipitate after discarding the supernatant. These purification steps were repeated several times until FITC was not observed in the supernatant. The final product was dialyzed against acidic water for ˜2 days and freeze-dried. The FITC Chitosan was transferred to a container with aluminum foil to protect it from light and stored at 4° C. until further use.

To study the gel-adhesive interface, the adhesion procedure describe previously was repeated using a 1:5 mixture of FITC:no-FITC chitosan as the bridging polymer. Interpenetration of the bridging polymer into the tissue was assessed using confocal microscopy (LSM710, Zeiss, Oberkochen, Germany). Sections were then mounted (Prolong™ Gold Antifade Mountant, ThermoFisher) and imaged using a confocal microscope for chitosan (FITC, excitation laser 488 nm) and collagen (polarized light).

### Hydrogel Swelling

Swelling ratio and rates for the gel and its dehydrated counterpart were studied. Both gel and film were originally cut to 15×20×1.7 mm³ dimensions. A 6-well plate was filled with 8 mL of DMEM and heated to ˜37° C. The gel and films (N=6) were placed in each well individually and their weights recorded over time until a swelling equilibrium was reached. The swelling ratio was then calculated as:

$$Swelling \% = \frac{{W}_{Swollen}}{{W}_{Regular}} \times 100$$

where W_{Regular} is the original mass of the as-synthesized gel.

### Gas Permeation Chromatography (GPC) of Alginate and Chitosan

The molecular weight distributions of the alginates were determined using size exclusion chromatography and software on the 1260 Infinity Multi-Detector GPC/SEC System. Two serial Viscotek A5000 300×8.0 mm columns were used for the size separation and all samples eluted off the columns using a 0.1M sodium nitrate with 0.5% (w/v) azide in double filtered Milli-Q water at a flow rate of 0.75 ml min⁻¹. All samples were dissolved in the above eluent between 2-3 mg/ml and injected at a volume of 100 ul. The system was calibrated under triple detection method using an Agilent polyethylene glycol standard (PL2083-2001).

For evaluation of chitosan MW, a buffer or 0.1M sodium nitrate, 0.01M sodium monobasic phosphate. with 0.5%. w/v azide, pH=3 adjusted with phosphoric acid in double filtered Milli-Q water at a flow rate of 0.75 ml min⁻¹ was utilized. The system was calibrated under triple detection using an Agilent pullulan kit (PL2090-0101).

### Fourier Transform Infrared Spectroscopy

To study the chemical structures of the different chitosan, Fourier Transform Infrared (FTIR) spectra was obtained of the dried polymers. All samples were measured as chitosan HCl salts. To prepare the samples, chitosan was dissolved in water and the pH was adjusted to 5.5 by adding 0.1M HCl. After a stable pH was obtained, the samples were frozen at −80° C. and lyophilized to eliminate water. FTIR spectra were recorded on the dry samples between 4000 and 400 cm⁻¹ on a Bruker ATR FTIR spectrometer.

### Rheology

Rheological measurements of the alginate and chitosan gel material (2% wt/vol) were carried out on a TA Instrument AR-G2 rotational rheometer. The measurements used a 20 mm diameter cone at 25° C. and plate geometry. Samples were placed on plate using a syringe at 75 ul volume and the upper cone was lowered to 0.060 mm gap distance. All samples were allowed to equilibrate for one minute before beginning the run. Flow curves were made using the flow step method with varying shear stresses, from 8.000E−3 Pa to 1.000 Pa over 600 secs at 25° C.

### Thermogravimetric Analysis

Chitosan thermogravimetric analysis was done using a Discovery Series TGA (S/N; TGA1-0031) and TRIOS software. A Platinum-HT (P/N: 957571.901) sample pan and Tzero Pan (P/N; 901683.901) were used. To analyze samples, platinum pans were heated using a heat gun to remove any residues. The following parameters were used: Mass Flow 40.0 mL/min; Initial Temperature 35.00° C.; Ramp 5.00° C./min to 250.00° C.; Isothermal 75.00 min. Samples were run in triplicate (n=3/group).

### Subcutaneous Implantation Model

All mouse experiments were conducted according to approved IACUC protocols. Balb/C mice at 20 weeks of age were anesthetized with isoflurane (2-2.5%) and given buprenorphine subcutaneously (0.5 mg/kg) for pain management. Hair on the mouse dorsum was removed with clippers and depilatory cream prior to adding three separate washes of betadine and ethanol. Animals were then transferred to the sterile field and placed beneath a separate sterile fenestrated drape. A small 6 mm incision was made through skin in the animal's back perpendicular to the midline, and then a subcutaneous pocket was created using scissors. Four separate gels (D=6 mm, th=1.5 mm) were then implanted subcutaneously, and the skin was closed with 4-0 Vicryl suture. Animals were monitored daily and gel swelling analyzed using HFUS (see below).

### High Frequency Ultrasound Imaging (HFUS)

HFUS (VisualSonics Vevo 770 and Vevo 3100; 35-50MHz) was used to evaluate gel swelling in vivo. Axial images (20 um axial resolution) were acquired that captured the skin and hydrogel. Images were used to quantify the thickness of the hydrogel and surrounding capsule after 0, 7, and 14 days. Images were analyzed using ImageJ (NIH).

### Statistical Analysis

Data normality was assessed with Shapiro Wilk tests (SPSS). One-way (time) or two-way (healing and TA implantation) ANOVAs with post hoc Student's t-tests were used.

### Chitosan Films Mechanical Properties

Pure shear tests were carried out to measure the matrix toughness. In brief, rectangular specimens (20×5 mm²) were tested in tension (Instron 3342, 50N load cell) at 100 mm/min. From the stress-stretch curves, the matrix maximum stretch, maximum stress, and fracture toughness were calculated¹³ using custom MATLAB code.

### Adhesion Testing

Adhesion energy was measured with 180° peeling tests (Instron 3342) under uniaxial tension (100 mm/min). For the tough adhesive, a tissue strip (˜75×11 mm²) was placed over the gel and compression was applied for 10 mins unless stated otherwise. For the tough adhesive, chitosan film (60×15 mm²) was placed between two gels and compression was applied for 30 seconds unless stated otherwise. Applied compression strain was kept at ˜5.5% in all cases. The back of TA was also bonded to a rigid polyethylene terephthalate (PET) film with cyanoacrylate (Krazy Glue), in order to limit deformation to the crack tip, and thus all the work done by the machine would be equal to the energy dissipated at the crack tip. The free ends of TA and the substrate were attached to acrylic pieces, to which the machine grips were attached. A mechanical testing system (Instron 3342, 50N load cell) was used to apply unidirectional tension, while recording the force and the extension. The loading rate was kept constant at 100 mm/min. The adhesion energy was two times the peak value of the ratio of the force and width.¹

### Lap Shear Testing

Tough adhesive samples containing a polyethylene mesh (Ethicon) were adhered to skin using chitosan (2% wt/vol). The samples were then apposed using a third tough hydrogel containing a mesh and a chitosan film. The peak force to separate the skin samples per 1 cm length of skin was recorded and compared to Dermabond Prineo® (Johnson and Johnson). The effects with and without the presence of blood was compared.

### Confocal Microscopy

The skin-gel adhesive interface and chitosan diffusion profiles for both the hydrated and dehydrated tough gels were studied by using confocal imaging and fluorescently labeled chitosan (FITC Chitosan). FITC Chitosan was synthesized by reacting fluorescein isothiocyanate (Sigma, 1245460250) with chitosan. Briefly, 1 g of ultrapure chitosan HCl was dissolved in 100 mL of 0.1M acetic acid and 100 mg of FITC was dissolved in 100 mL of anhydrous methanol at 1.0 mg/mL in separate flasks. The FITC solution was then slowly added to the chitosan solution with continuous stirring. After ˜3 h, the reaction was quenched by slowly adding NaOH (0.5M) to increase the pH to ˜10, precipitating the fluorescently labelled chitosan. The solutions were then centrifuged, and dIH2O was added to the precipitate after discarding the supernatant. These purification steps were repeated several times until FITC was not observed in the supernatant. The final product was dialyzed against acidic water for ˜2 days and freeze-dried. The FITC Chitosan was transferred to a container with aluminum foil to protect it from light and stored at −4° C. until further use.

To evaluate the gel-film interface and morphology, the adhesion procedure described previously was repeated using a 1:5 mixture of FITC:no-FITC chitosan as the bridging polymer and gel synthesis with 0.1% nile blue acrylamide (#25395-100, Polysciences). Whole samples were used or sagittal sections generated with a cryostat (Leica) were then mounted (Prolong™ Gold Antifade Mountant, ThermoFisher). The interface of the chitosan film into the gel was assessed using confocal microscopy (LSM710, Zeiss, Oberkochen, Germany). Samples were imaged using a confocal microscope for the tough hydrogel (nile blue, excitation 633 nm) and chitosan (FITC, excitation laser 488 nm).

### Scanning Electron Microscopy

Elemental mapping was performed on native (uncoated) samples under low vacuum conditions (20 Pa) using a Tescan (Brno, Czech Republic) Vega GMU scanning electron microscope equipped with a Bruker XFlash 5030 dual-detector EDS system. Each of the 512×512 elemental maps was acquired at an acceleration voltage of 20 keV and at a 15 mm working distance.

### Evaluation of Zeta Potential

Samples of chitosan (85 and 95% degree of deacetylation), acrylamide, alginate, and alginate-AAM were dissolved in water and/or HBSS. The resulting solution was analyzed with a particle size analyzer (Malvern zen3600) for zeta potentials.

### Burst Pressure Testing

Standard tests (ASTM F2392) were completed for assessment of burst strength. A 15 mm diameter tough adhesive with and without attachment of a chitosan film to its back was evaluated. The pressure was applied by pumping air using a syringe pump (Harvard Apparatus PHD 2000 Dual Syringe Pump) at a rate of 2 mL/min through a 3 mm defect at the bottom of the sample. During measurement, the pressure was recorded, and the burst pressure was identified when a burst occurred.

For dynamic tests, a peristaltic pump was utilized. Aortic tissue (Sierra Medical) was carefully prepared and attached to liquid tight clamps. A 1 cm longitudinal defect was made and was let repaired or not repaired with suture (4-0 Prolene) followed by application of the tough adhesive with or without a chitosan film. A water/cornstarch mixture was used to mimic blood. Pump rates (80-100 cycles/min) were completed and the pressure was controlled with a clamp on the distal side. Pressures were ramped until failure occurred.

### Cooling Effects of Gels

Tough hydrogels and TenderCare® samples (15 mm×15 mm×1.5 mm) were cut and placed on the palms of gloved hands (IRB Exempt) for 1-minute. The temperature before and after placement was recorded with an IR camera (make/model).

### High Frequency Ultrasound Imaging

High frequency ultrasound imaging (Visualsonics, VEVO 3100, 50MHz transducer) was used to evaluate placement of the gels over bovine tendon and nerve tissues.

### Release of 5FU from Tough Hydrogel/Chitosan Film Patch

Chitosan was dissolved at 2% wt/vol in HBSS at varying pH levels (˜3.5-5.5). 1% 5FU (drug) were then incorporated into the chitosan solution and allowed to mix overnight. The chitosan liquid mix was then poured into a glass mold and was placed in an oven at 64C for 4 hours to dry into a film. The film was then removed from the glass and placed in a sealed bag prior to use. To quantify the amount of 5FU released, a standard curve of 5FU was generated, with absorbance evaluated at 266 nm and 750 nm on a plate reader. To conduct the experiment, several gel designs were prepared and sampled daily for drug release over 7 days. The first design involved a chitosan film placed between two 8 mm diameter tough gels (alginate/acrylamide). The second design consisted of a chitosan film on a single 8 mm diameter tough gels. The third design utilized a chitosan film sandwiched between two VHB elastomers. The fourth design utilized a chitosan film sandwiched between a tough hydrogel and VHB elastomer. Lastly, a lower pH chitosan film (pH 3-3.5) was sandwiched between two 8 mm diameter tough gels. The buffer used for the experiment was HBSS, with 500 microliters per sample, and the samples were placed in eppendorfs. The experiment was performed in triplicate at a temperature of 37° C. while shaking. Each day, 500 microliters of buffer were removed and stored, followed by the complete replacement of the HBSS buffer to establish sink conditions.

### Results

### Chitosan Enables Rapid and Strong Tissue Adhesion

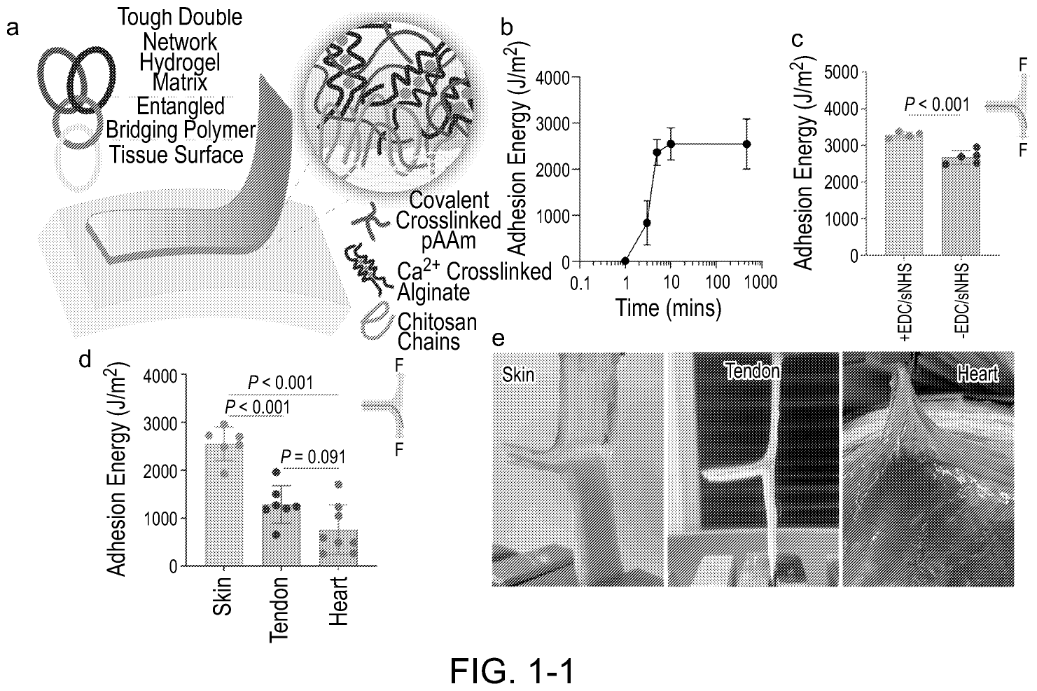

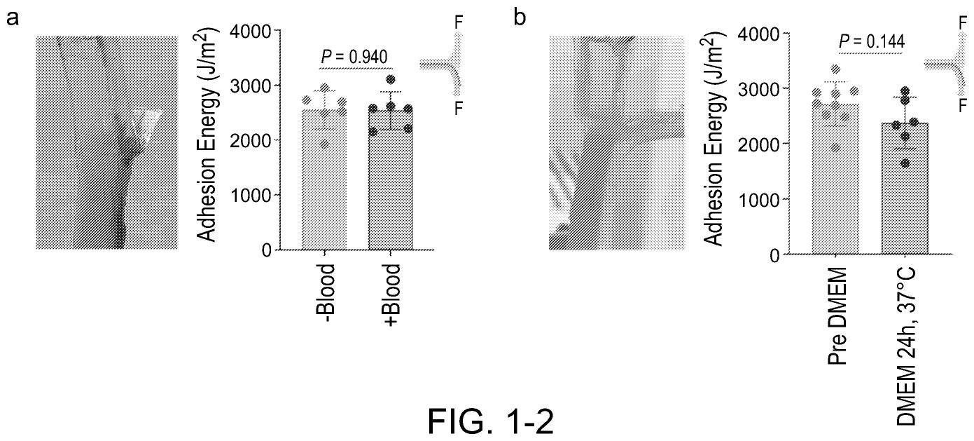

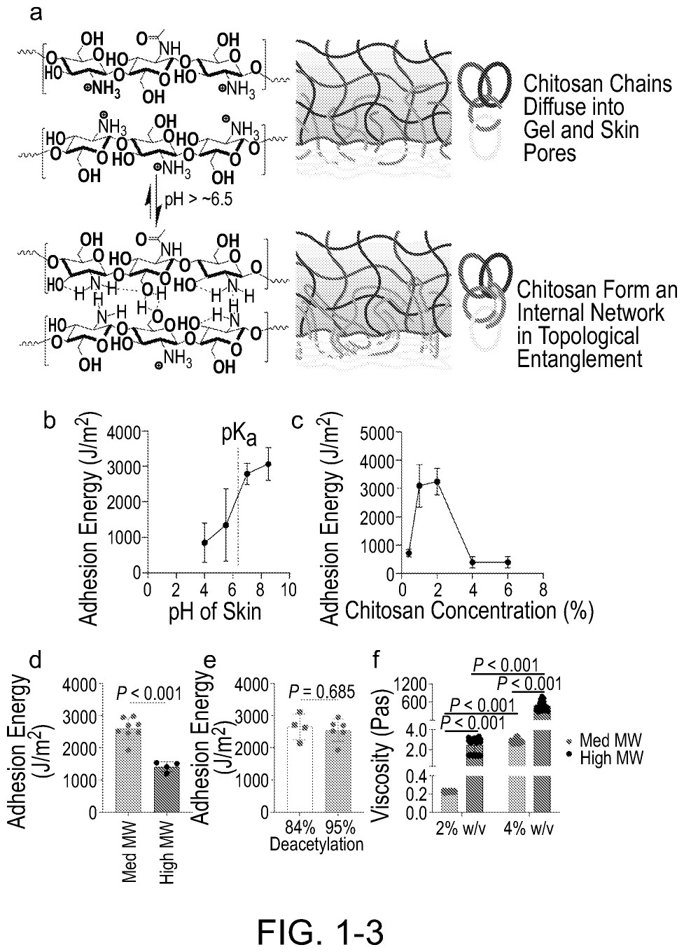

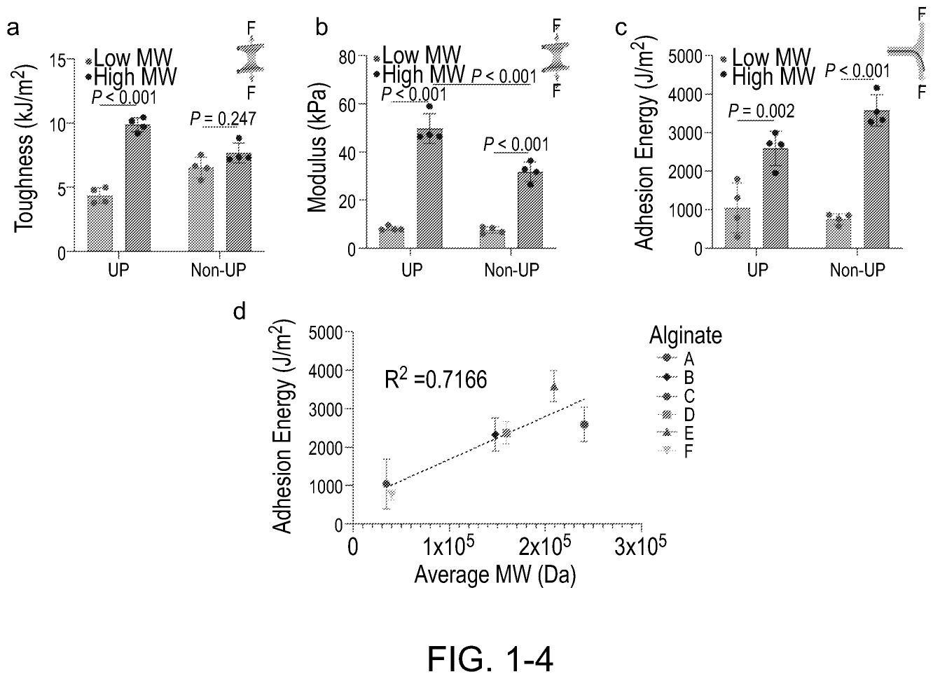

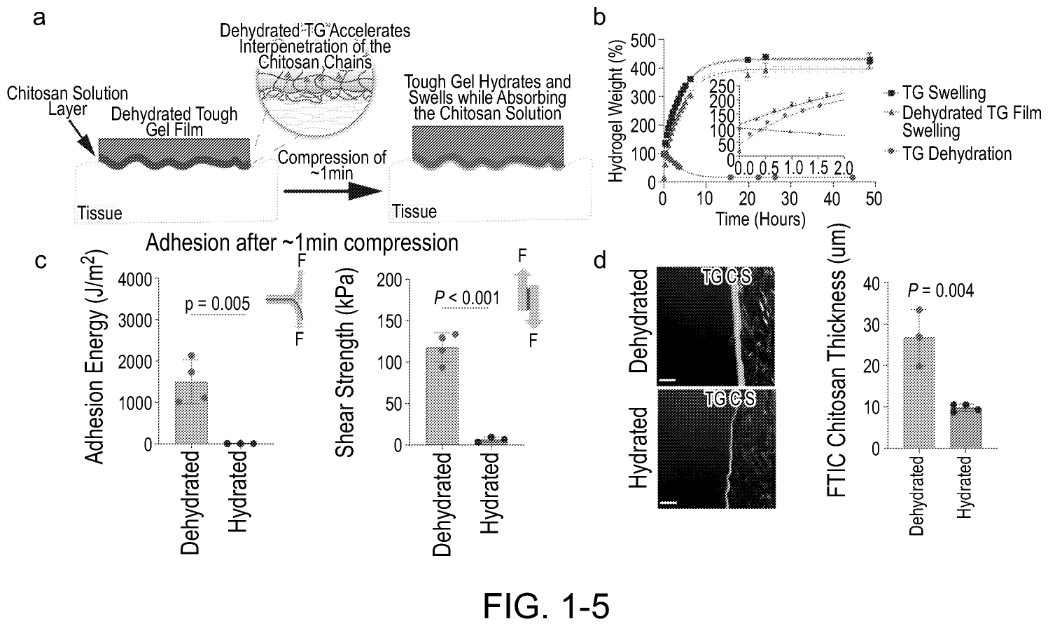

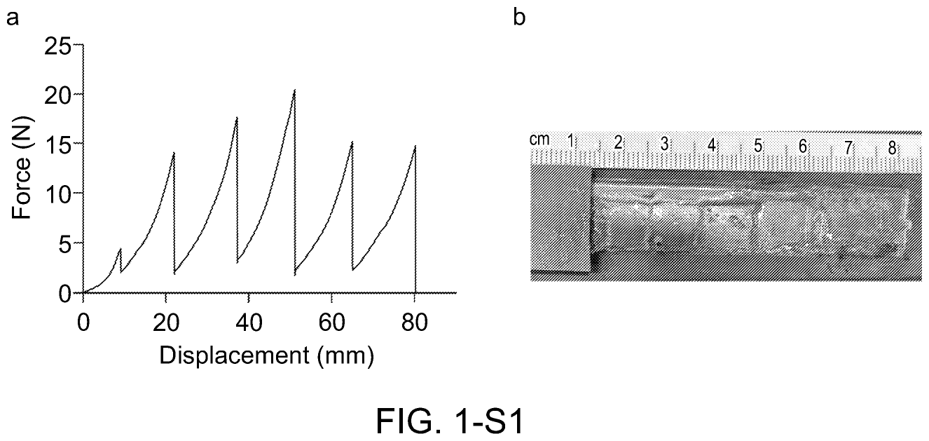

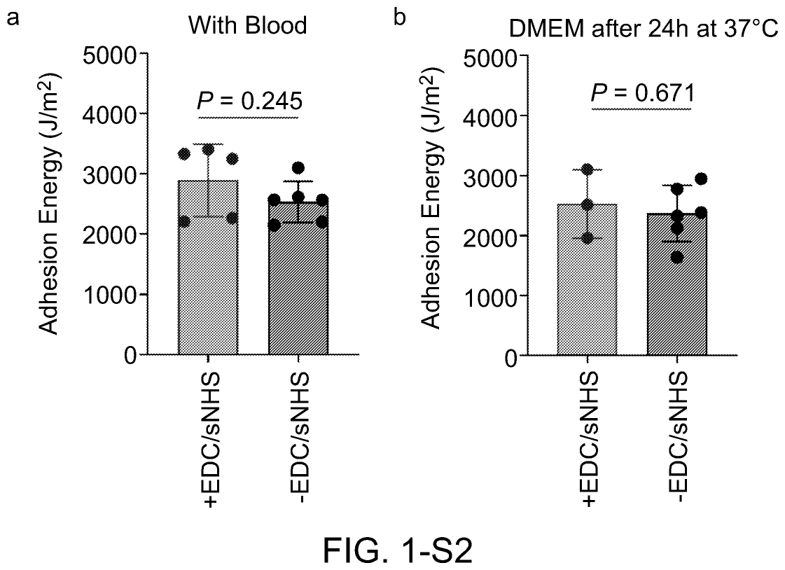

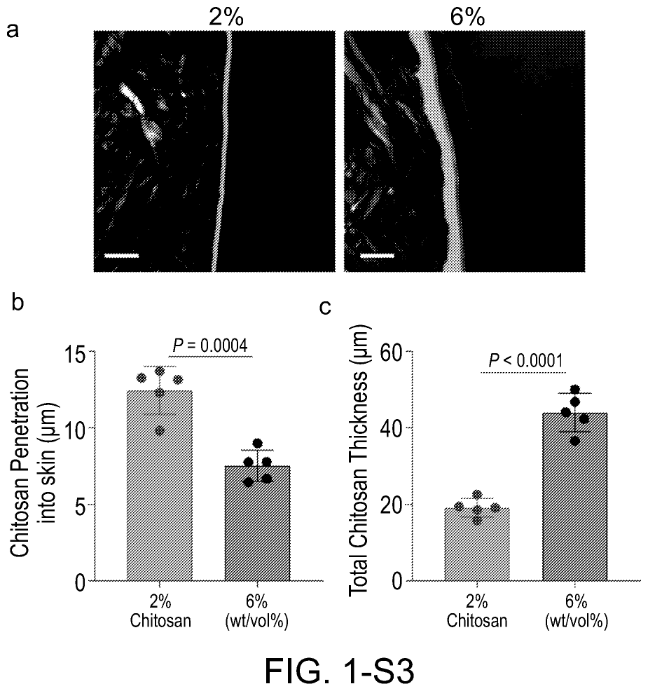

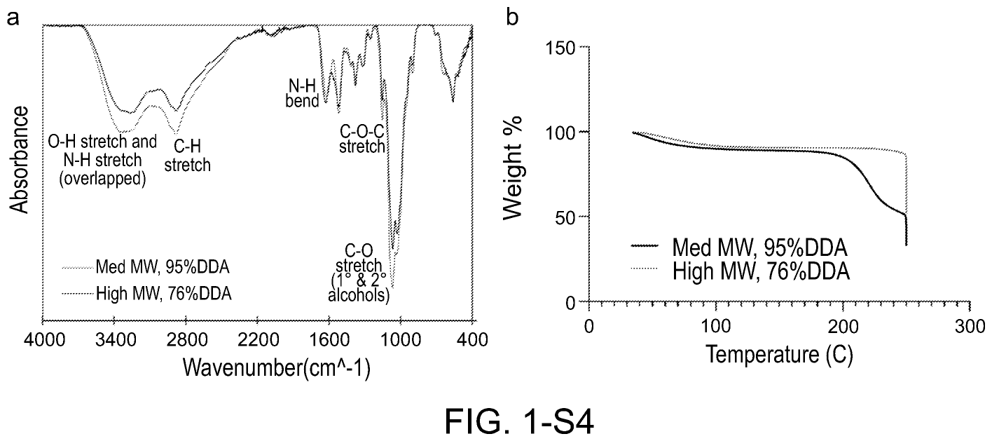

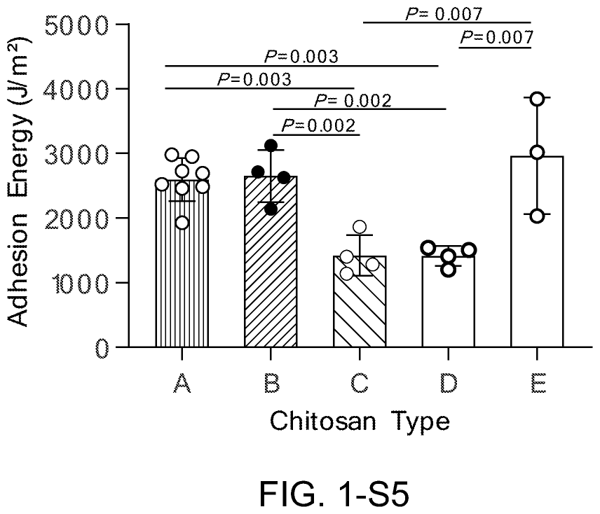

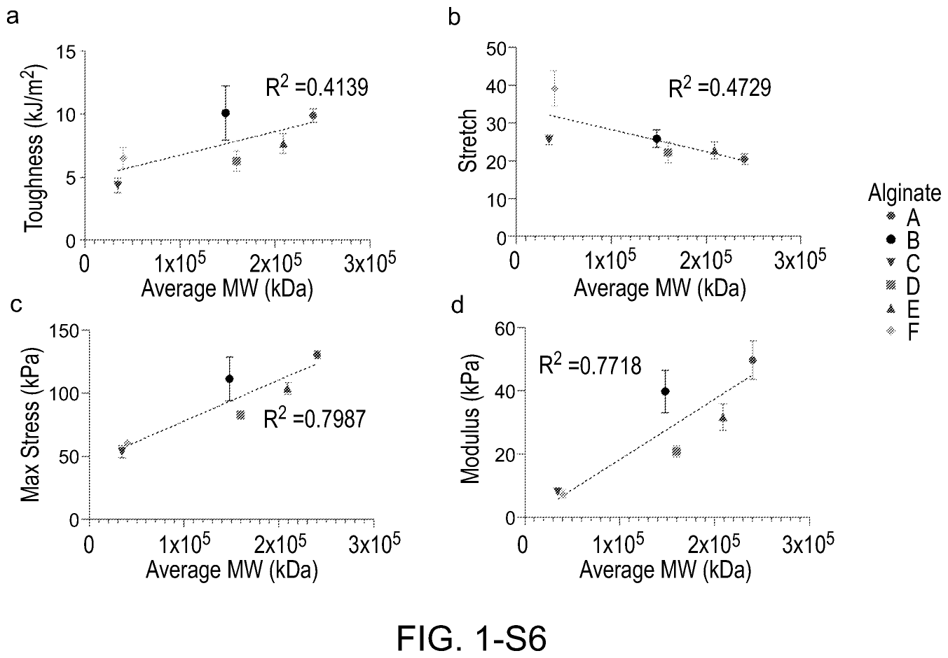

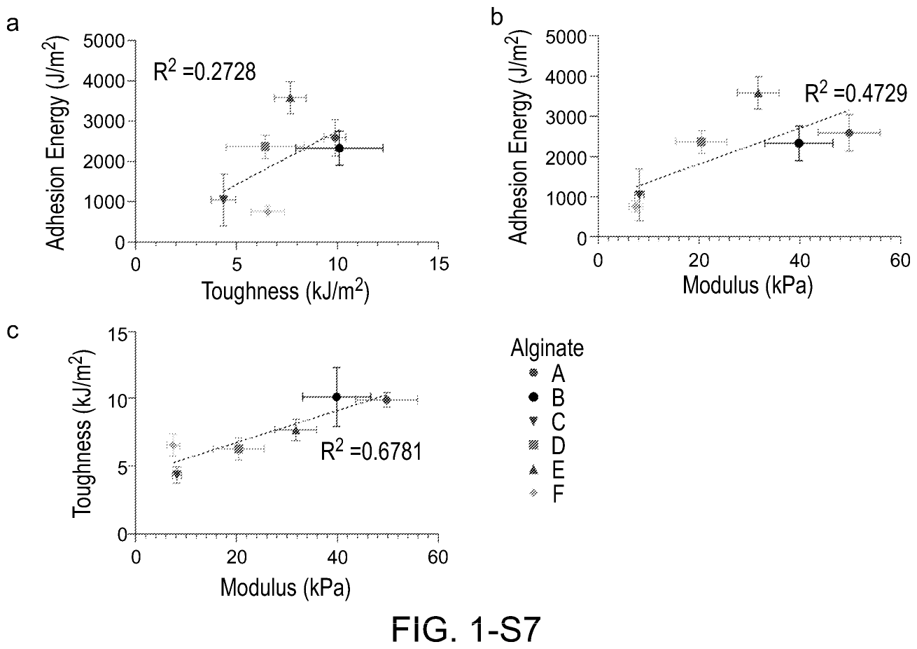

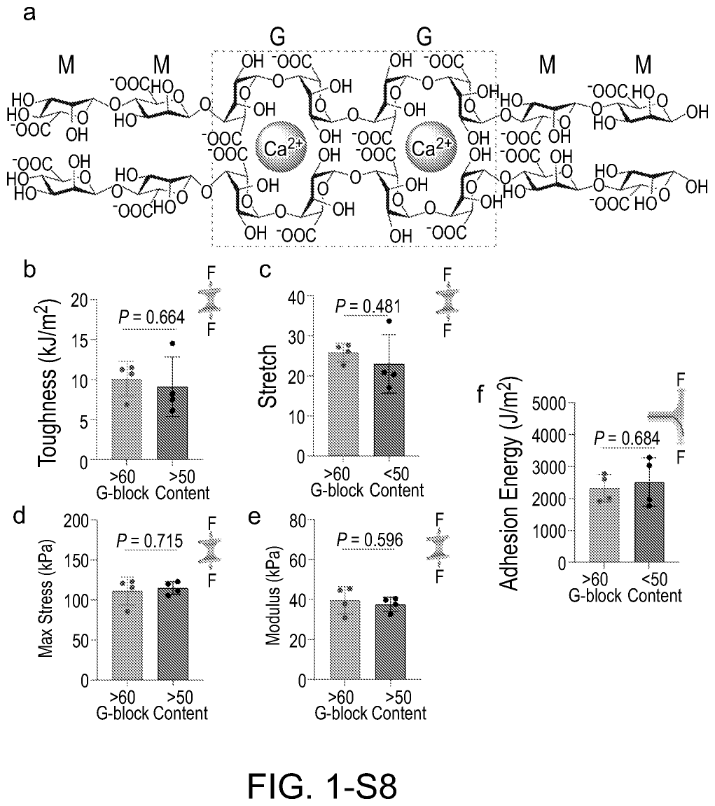

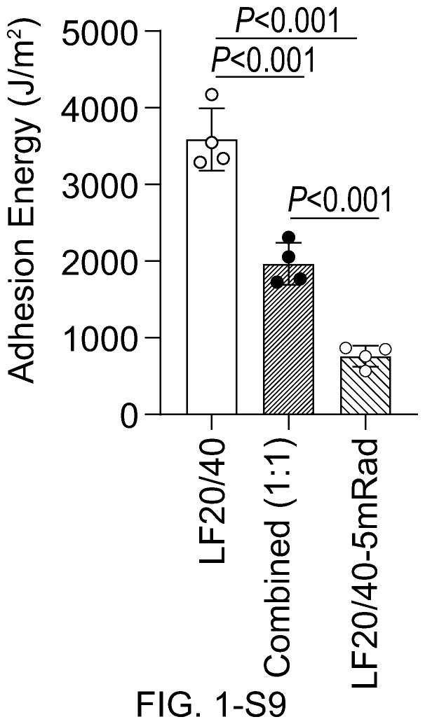

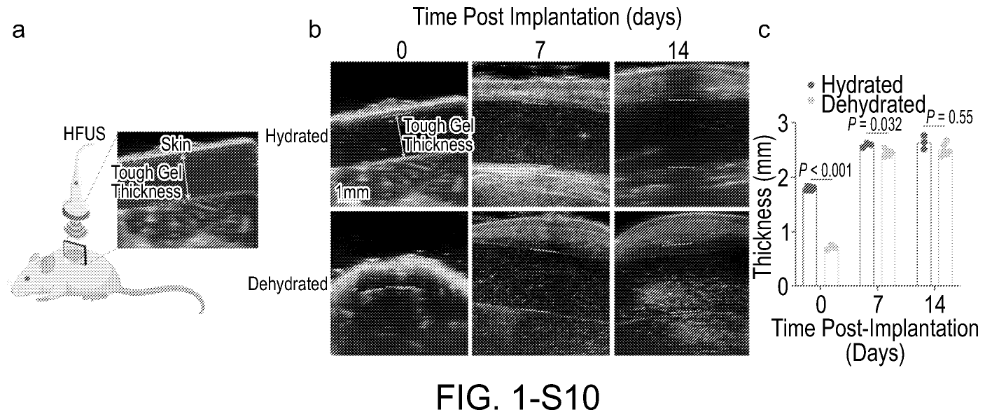

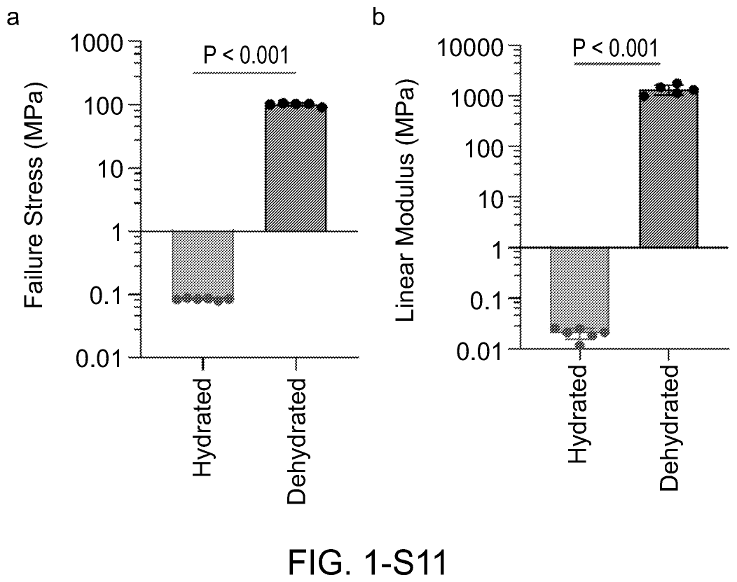

The ability of chitosan to mediate adhesion to skin tissue was first investigated (FIG. 1-1a). After application of the hydrogel and bridging chitosan-only adhesive to skin tissue, the adhesion energy increased rapidly and exceeded 1000J/m² by about 3 minutes, and 2000J/m² by about 10 minutes before reaching a steady state (FIG. 1-1b). Peak forces during adhesive removal exceeded 20N, and periodic cohesive hydrogel failure during removal was observed (FIG. 1-S1). Notably, strong adhesion due to chitosan did not require the addition of coupling reagents mediating covalent bond formation, as addition of EDC and sNHS led to only a moderate increase in adhesion energy (FIG. 1-1c). In addition to skin, strong adhesion using chitosan alone was also observed with tendon and heart tissue (FIG. 1-1d).

### Adhesion is Stable and Robust to Blood

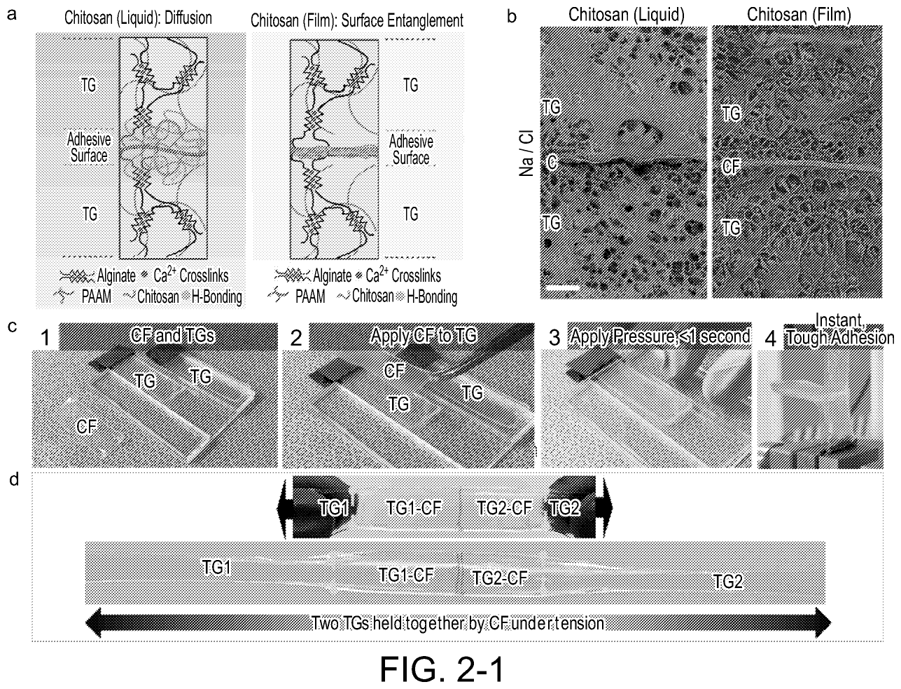

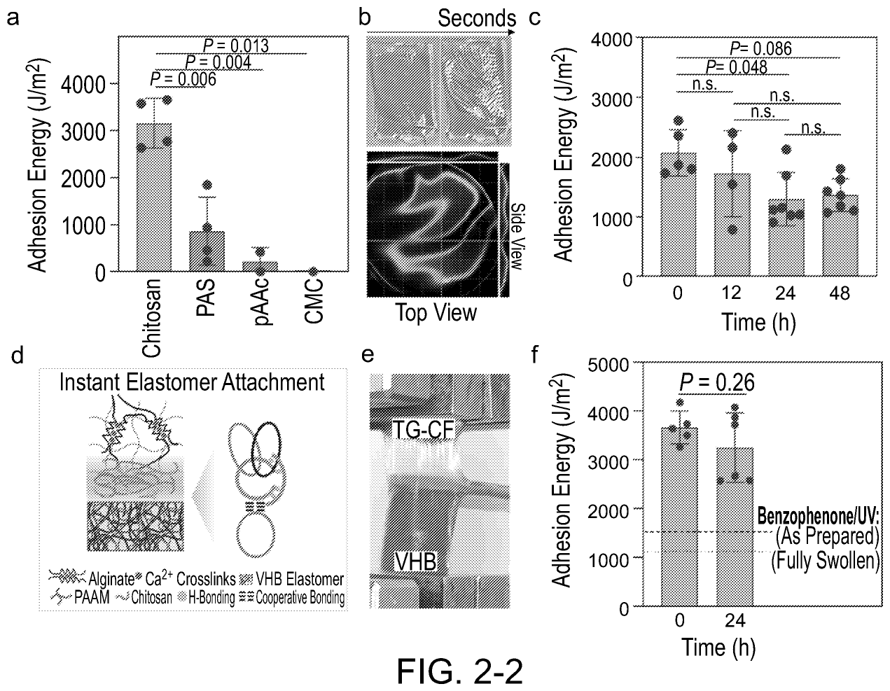

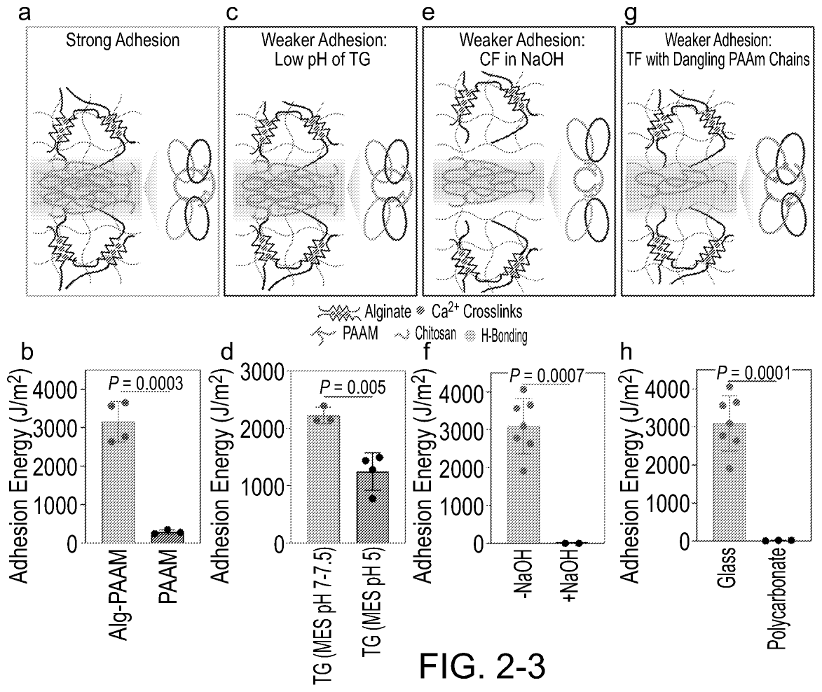

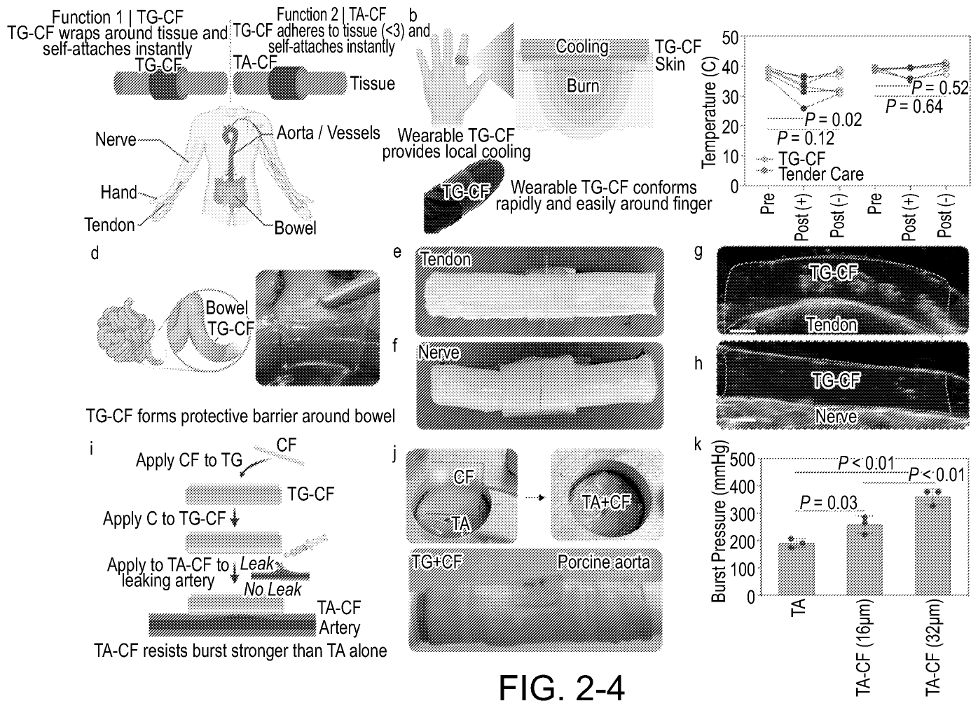

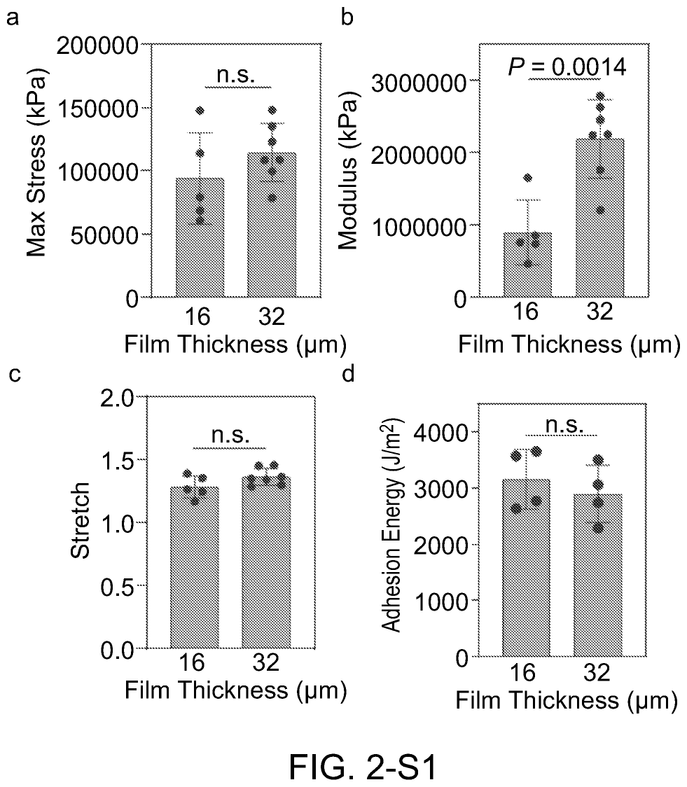

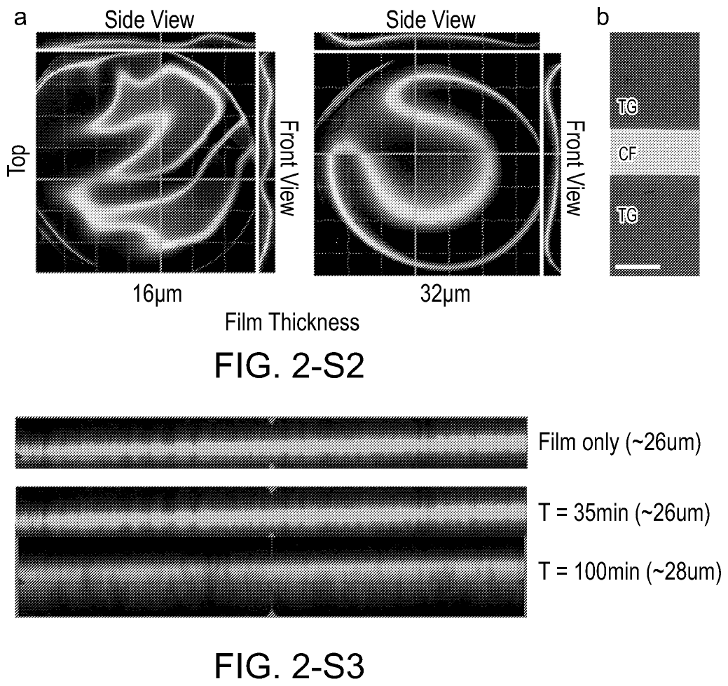

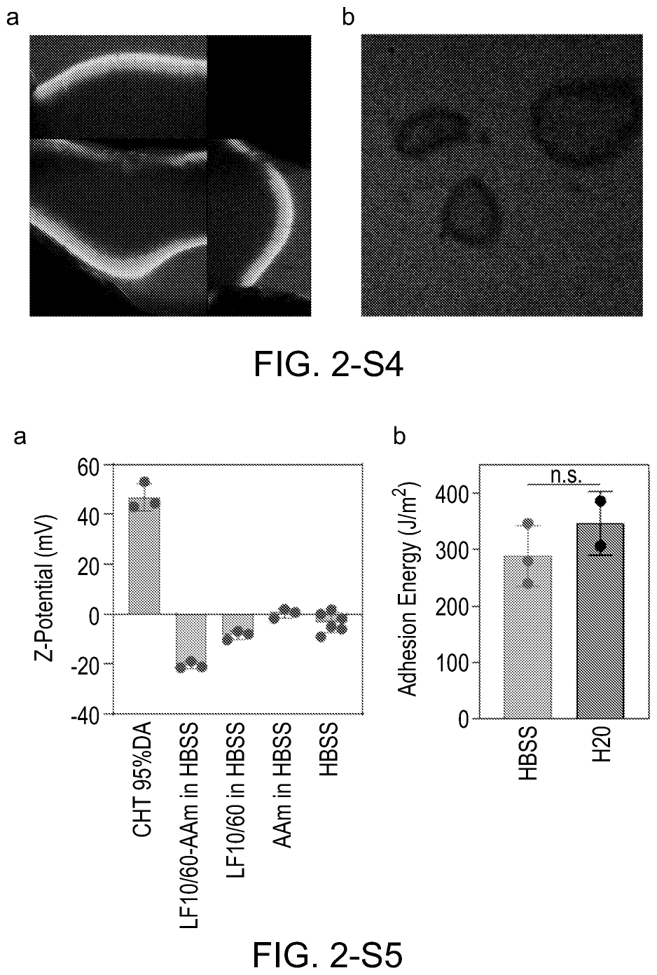

To examine the versatility of this adhesive approach, testing was completed comparing performance with and without addition of blood and following incubation in Dulbecco's Modified Eagle Medium (DMEM) at 37° C. for 24 h to achieve a fully swollen state. Non-covalent adhesion enabled strong adhesion to skin tissue, regardless of the presence of blood (FIG. 1-2a) and it occurred independent of addition of covalent coupling reagents (FIG. 1-S2a). Adhesion to skin was maintained following incubation in DMEM for 24 h at 37° C. (FIG. 1-2b) and was again independent of the addition of covalent coupling agents (FIG. 1-S2b).

### Adhesion Strength Depends on pH, Bridging Polymer Concentration, and Viscosity

Previous studies have suggested pH-dependent topological adhesion between two hydrogels^{[20]}, and several factors were next investigated to explore if this mediates the strong tissue adhesion observed here (FIG. 1-3a). The adhesion energy was increased with increased pH of skin, beyond the pKa of chitosan (FIG. 1-3b). The adhesion energy was also dependent on the concentration of chitosan, showing a maximum at approximately 2 wt/vol % (FIG. 1-3c). Higher concentrations of chitosan resulted in significantly lower penetration into skin tissue and a thicker interfacial chitosan layer (FIG. 1-S3). Tough adhesion was affected by the MW of chitosan (FIG. 1-3d), but not the degree of deacetylation (FIG. 1-3e). Although similar bands in the FTIR spectra of ultrapure and non-ultrapure chitosan were observed, differences in thermal decomposition were detected (FIG. 1-S4) in concert with reported differences in MW/purity and degree of deacetylation (Table 1-S1). However, the chitosan molecular weight of these chitosan samples resulted in significantly different viscosities, separated by at least one order of magnitude (FIG. 1-3f). Topological adhesion with other chitosans with different molecular weights, purity, and degrees of deacetylation was also tested and similar trends were observed (FIG. 1-S5 and Table 1-S1).

Gels with High Molecular Weight Alginate Facilitate Stronger Topological Adhesion

The effect of different alginates (Table 1-S2 and FIG. 1-S4) on hydrogel mechanics and adhesion energy were next evaluated to address the hypothesis that topological entanglement may be affected by molecular weight of polymers in the dissipative matrix. Hydrogel toughness was affected by alginate molecular weight (FIGS. 1-4a and 1-S6), while modulus was affected by both alginate molecular weight and purity (FIG. 1-4b). Additionally, the adhesion energy was affected by alginate molecular weight regardless of whether ultrapure or non-ultrapure polymers were used in gel fabrication (FIG. 1-4c). Indeed, the adhesion energy was strongly and positively correlated with alginate molecular weight (FIG. 1-4d). The adhesion energy was also correlated to hydrogel toughness (R²=0.27) and modulus (R²=0.47) (FIG. 1-S7). Hydrogel toughness was correlated to hydrogel modulus (R²=0.68) (FIG. 1-S7). Alginates with higher G-content, the residue involved in ionic crosslinking, did not result in higher toughness, stretch, maximum stretch, modulus, or adhesion energy (FIG. 1-S8). Although the combination of high and low molecular weight alginates has been suggested to result in elevated toughness^{[9]}, adhesion energies did not follow this similar trend, with the highest observed in gels of higher molecular weight alginates only (FIG. 1-S9).

### Dehydrated Gels Generate Near Instantaneous Adhesion to Tissues by Accelerating Interpenetration and Entanglement of the Chitosan Chains.

It was next examined whether adhesion could be accelerated by promoting rapid infiltration of chitosan into dehydrated gels. Chitosan is added to the hydrogel prior to application on tissue. After chitosan is applied on the hydrogel surface it diffuses into the dissipative matrix faster when it is in the dehydrated state (FIG. 1-5a). While hydrogels require approximately 24 h to dehydrate, they rehydrate to their original mass within ˜30 minutes and then continue to swell over incubation in a large pool of buffer (FIG. 1-5b), highlighting the fast hydration process. Dehydrated gels demonstrated more rapid, initial swelling than a gel that was not initially dehydrated (3× swelling rate over first 12 minutes). A similar final swelling equilibrium state was observed in vitro, regardless of initial gel dehydration state. Implantation of dehydrated gels also led to similar in vivo swelling as non- dehydrated, implanted gels over 2 weeks, but the swelling of both types of gels was considerably lower than observed in vitro (FIG. 1-S10). Dehydrating the tough gels to a film before adhesion also resulted in a 50,000-fold increase in linear modulus and a 1,000-fold increase in failure stress (FIG. 1-S11). Strikingly, the adhesion energy of the dehydrated tough gel exceeded 1000J/m² within about 1 minute of application and shear stresses exceeding 100 kPa, in contrast to much lower values with the hydrated gels (FIG. 1-5c). Confocal imaging of FITC labeled chitosan revealed that there was a noticeably greater chitosan penetration into dehydrated gels within 1-minute, as compared to hydrated tough gels (FIG. 1-5d).

### Chitosan Films Generate Instant Tough Adhesion Between Gels

In contrast to previously described liquid-based bridging polymers, that require diffusion into the adherends and subsequent entanglement to generate adhesion (8.1), film-based dry bridging polymers are proposed to generate rapid adhesion. Specifically, dry chitosan films are used to create adhesion through localized and dense surface entanglement and H-bonding to tough alginate-polyacrylamide hydrogels, a dual network hydrogel with extraordinary material toughness (14.1) (FIG. 2-1a). Elemental mapping via energy dispersive spectroscopy (EDS) (FIG. 2-1b) revealed a distinct localized chitosan film (CF) region at the interface between the adherent tough gels (TG) following application. Instant, tough adhesion was achieved between hydrogels under standard peel test conditions (FIG. 2-1c) and when pulled in tension (FIG. 2-1d).

### Chitosan Films Generate Stable Adhesion

Several polymer films based on bridging polymers with different functional groups like amines and carboxylic acid groups were next tested, including poly(amino styrene) (PAS), poly(acrylic acid) (PAAc), and carboxymethyl chitosan (CMC). Chitosan-based films generated instant (within 1 second) tough adhesion between hydrogels and achieved the highest adhesion energy (>3× other films) (FIG. 2-2a), potentially due to their strong film mechanics in the dry state (FIG. 2-S1a-c) and wrinkling of the films on the surface (FIG. 2-2b) which increases the surface area in contact between the adherends while extending the crack propagation path and energy dissipation zone (15.1, 16.1). PAS (pKa ˜4.5), another polymer with a high density of primary amines achieved weaker adhesion compared to chitosan. An attempt to test other bridging polymer films with amine groups like poly(allylamine) (PAA) and polyethyleneimine (PEI) was unsuccessful as these polymers did not generate films after solvent evaporation possibly because of their C-C flexible backbone, small side groups, or molecular weight. PAAc (pKa ˜4.5) and CMC (pKa ˜2-4) (17.1), both bearing carboxylic acid groups capable of H-bonding, generated films but did not demonstrate any adhesion. While carboxylic acids become charged and lose the ability to form H-bonds when the pH>pKa, amine groups lose their charge and gain the ability to form H-bonds when the pH>pKa. All gels were made in HBSS buffer (pH ˜7.4) and only chitosan (pKa ˜6.5) was able to generate strong adhesion under these conditions, suggesting that the bridging polymer used for dry films must be able to form H-bonds and strong chain-chain interactions at a specific pH.

The adhesion energy generated between TGs was not affected by chitosan dry film thickness (FIG. 2-S1d), despite differences in wrinkling patterns of the films after placement onto hydrogel surfaces (FIG. 2-S2). Serial confocal imaging revealed stable CF thickness after application through 100 min, suggesting that the CFs remain as a distinct layer between gels and do not undergo significant swelling (FIG. 2-S3). The adhesion with these films was so strong that after peel testing, residual PAAm was observed on the bridging surface due to cohesive failure of the gel matrix (FIG. 2-S4a), leaving local surface defects in the TG (FIG. 2-S4b). Following incubation in DMEM at 37° C. for 12 h, 24 h, and 48 h, high adhesion energy was maintained, although there was some decline in adhesion strength over time due to cohesive failure, highlighting its sustained performance in a challenging scenario (FIG. 2-2c).

Chitosan films enabled rapid, robust attachment of the TG-CF to elastomer (VHB, 3M) potentially through cooperative bonding between the dense film and the two adherends (FIG. 2-2d). Attachment of the TG-CF to VHB produced instant strong adhesion (up to >4000J/m²) (FIG. 2-2e), without the need for chemical treatments or use of ultra-violet (UV) light for crosslinking, which may require an excess of 30 minutes for bonding to occur (18.1). Adhesion was stable and remained unchanged following incubation in culture medium for 24 h (FIG. 2-2f).

### Tough Adhesion is Governed by pH and Entanglement, but not Electrostatics

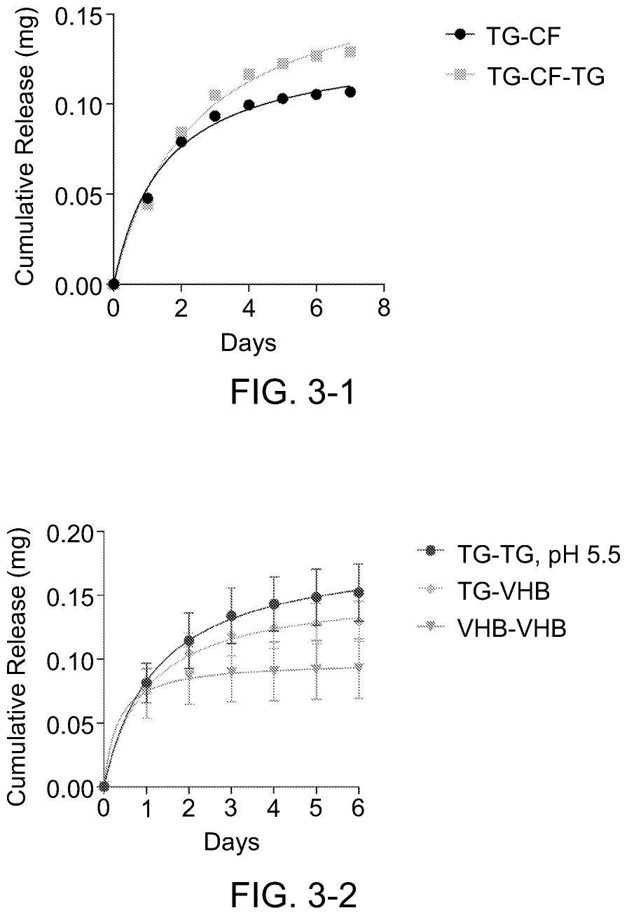

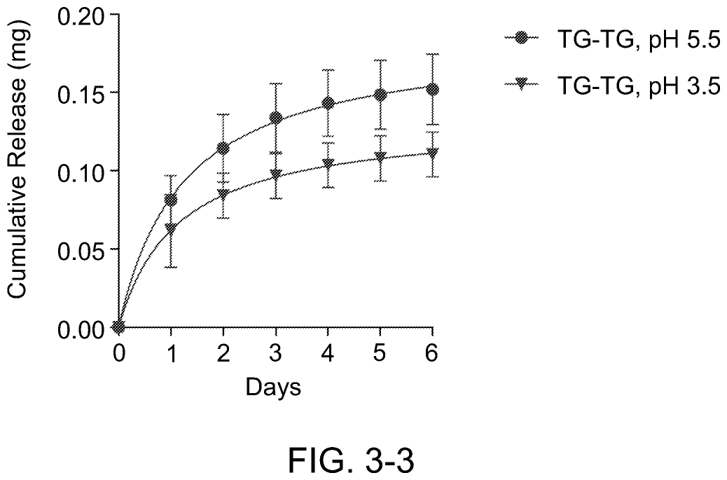

To evaluate the mechanism of CF adhesion to TGs, the potential contribution of electrostatic-mediated adhesion was first investigated. Although alginate carries a negative charge and chitosan carries a positive charge at neutral pH (FIG. 2-3a), interfacial toughness comparable to that of liquid chitosan after 24 h (8.1) was generated instantly in net neutral PAAm-only gels using CF (FIG. 2-3b). Cohesive failure was observed with a peel test (FIG. 2-S5). This finding highlights that adhesion energies are dependent on gel mechanics and suggests that this strategy could be extended to other hydrogel compositions. Previous studies have shown that hydrogels with lower pH reduce interfacial toughness when using liquid chitosan as a bridging polymer, likely because of less deprotonation of amine functional groups (8.1, 10.1). This represents a practical challenge since human tissues and environments vary in pH (19.1). Thus, whether these effects were also present in the dry film format were tested. Decreasing the pH of the Alg-PAAm gels decreased adhesion energy; however, adhesion strength was still exceptional (>1000J/m²) (FIG. 2-3c,d) compared to commercial adhesives. This suggests that pH of the adherends still influences adhesion when utilizing the dry chitosan films (>pH 6.5 ideal), but that the adhesion energy remains very high even in variable pH conditions. Deprotonating the chitosan film through immersion in 1M NaOH inhibited subsequent adhesion between gels (FIG. 2-3e,f). It is believed that deprotonation of the films before application creates strong interactions between the chitosan chains, preventing them from interacting with the substrate. This further suggests that H-bonding with or within the adherends is part of the adhesion mechanism. In addition to pH and electrostatics, the importance of surface entanglement to generate adhesion was evaluated by using gels with different surface topologies (FIG. 2-3g). Gels cast on hydrophobic oxygen permeable polycarbonate (20.1, 21.1), which increases the presence of dangling PAAm chains in the double network by interfering with the free radical polymerization process (22.1), demonstrated significantly lower adhesion (FIG. 2-3h).

### Self-Adhering TGs Enable Applications in Biomedicine

Since dry chitosan films enable unprecedented instant adhesion between Alg-PAAm and PAAm hydrogels compared to previous studies (1.1-5.1, 8.1, 10.1, 23.1), several proof-of-concept examples for their applications as fast self-adhering biomaterials were explored when integrated with a TG or TA (FIG. 2-4a). The TG could easily wrap around cylindrical objects, such as a finger (FIG. 2-4b), demonstrating its favorable handling characteristics that would be practical in surgical or other clinical scenarios. Due to the higher thermal conductivity of water in the gels, its capacity to provide local tissue cooling were explored. Following application of the hydrogel on the surface of human palms, the temperature of the skin was found to decrease unlike a standard of care hydrogel (e.g., Tender Care®) (FIG. 2-4c), likely due to elevated water content in the tough gel. This could have clinical implications for patients suffering burn injuries, by maintaining regional cooling, while preventing rapid fluid loss through the damaged barrier.

Fibrotic adhesions following surgery or injury are common and can have devastating consequences, remaining an unmet clinical need. While commercial technologies such as Seprafilm® (Baxter, Deerfield IL) provide a hydrogel barrier, they have been limited by their mechanical properties and poor function in liquid medium, and are contraindicated in many operations where postoperative adhesions are a primary concern, such as bowel anastomoses, thereby limiting its utility. Since the TG alone does not adhere to tissues, the self-adhering TGs using CFs may be useful for internal applications that require gel-to-gel adhesion but non-adherence to underlying tissue, therefore maintaining tissue planes while also promoting physiologic gliding (i.e., anti-adhesion) with adjacent structures. For applications that require wrapping a TG around a body part, instant gel-to-gel adhesion would be a critical feature enabled by fast adhesion and CFs. The TG+CF easily wrapped around organs such as bowel (FIG. 2-4d), tendon (FIG. 2-4e,g), and peripheral nerve (FIG. 2-4f,h) highlighting their potential application as anti-adhesion barriers with surrounding tissues (24.1).

The ability of CFs to enable TGs as internal sealants for fluid leaks in the body by providing mechanical reinforcement was explored ex vivo. Whether addition of the CF could serve as a clinically relevant and biocompatible backing to increase peak burst pressures of TAs (FIG. 2-4i) were also investigated. The CF was applied to a TA adhered to the surface of a swine aortic arteriotomy model and withstood thousands of simulated cardiac cycles highlighting a simple strategy to increase its burst pressure resilience (FIG. 2-4j). Peak burst pressures were significantly increased with addition of a CF, and this was further increased when using thicker CFs (FIG. 2-4k), which displayed elevated modulus.

Taken together, this work presents a simple strategy to generate instant tough adhesion between alginate-polyacrylamide hydrogels using CFs. Strong film mechanics and adhesive polymer films with pKa around physiological pH are key for achieving adhesion through interfacial topological entanglement and H-bonding. These findings and the favorable handling characteristics of this technology supports clinical applications and has important implications for designing composite hydrogels, and interfacing devices with the human body where fast and robust adhesion between gels is required.

Slow Release of 5-FU (Fluorouracil) from the Chitosan Film

In order to further study the application of the TG/chitosan film, a study was completed which measures the release from 5-FU from a chitosan film in a saline solution over time, which mimicked physiological conditions. In order to complete this test, the chitosan film was prepared which was infused with 5-FU, which was used in all experiments. Two different designs were tested, the first being a single chitosan film adhered to a single hydrogel. This was compared to the second design, which was a single chitosan film sandwiched between a first and a second hydrogel. The comparative 5-FU release for these designs in a saline solution at pH 5.5 can be seen in FIG. 3-1, which demonstrated that the “sandwich” design released more 5-FU (by mass) over the 7 day testing period.

With the aim of expanding upon the potential “sandwich” design, different materials for the “sandwich” design were tested. First, the single chitosan film was placed between a first and a second VHB elastomer. Second, the single chitosan film was placed between a hydrogel and a VHB elastomer. These two alternative designs were compared to the initial “sandwich” design (chitosan film in between hydrogels) in a saline solution at pH 5.5. The results of this experiment can be seen in FIG. 3-2, which demonstrated that the initial sandwich design at pH 5.5 achieved the highest 5-FU release over time. Finally, the initial sandwich design in a saline solution of pH 5.5 was compared to the same design in a saline solution at pH 3.5. The result of this experiment can be seen in FIG. 3-3, which shows that the pH 5.5 solution demonstrated the highest 5-FU release over time.

### Discussion

This study investigated the potential adhesion mechanisms of chitosan-mediated, alginate-pAAm tough hydrogels adhesion to tissues. The study demonstrates that strong topological adhesion to tissues can be achieved using tough hydrogels, without the need of covalent bond formation.^{[1, 13]} Adhesion strengths achieved were >2000J/m², higher than typically employed hydrogel adhesive materials which rely on direct bond formation with tissues (e.g., cyanoacrylates, catechol, aldehyde, or activated NHS carbonyls).^{[1, 13, 21]} Tissue adhesives relying on non-covalent interactions are typically weak and not stable.^{[18]} However, use of a combination of a hydrogel dissipative matrix with high molecular weight alginate and a dilute chitosan solution achieved fast, strong, and stable adhesion through physical non-covalent interactions mediated by the bridging polymer. Unprecedented levels of adhesion were shown to be stable, physiologically relevant, and occur independent of coupling reagents while remaining unperturbed after placement in large excess of buffer and bloody settings.

The adhesion mechanism observed in this study was mediated by the chitosan bridging polymer. Chitosan has long been used as an adhesive because of its biocompatibility and pH responsiveness.^{[22]} However, without a tough matrix, peak adhesion stress in the absence of covalent bonds is typically <100 kPa.^{[23]} The results disclosed herein support the previously proposed pH dependent adhesive properties of chitosan; deprotonation of its amine groups drive strong interchain interactions by cooperative H-bonding. It thus forms a chitosan network within the gel and tissue.^{[20]} As chitosan's pKa is ˜6.5, tissues (pH>6.5) can induce its gelation upon deprotonation of the chitosan chains, generating an internal interpenetrating network. Additionally, since the pH of blood (˜pH 7) is above the pKa of chitosan, it is possible that the chitosan amines will begin to deprotonate, triggering the formation of a chitosan entangled network prior to interacting with the tissue. However, application of compression displaces some of the blood and allows the hydrogel to contact the underlying tissue. In this setting, it is unlikely that there is sufficient time for significant network formation within the chitosan solution. For tissues with lower pH, the addition of coupling reagents to mediate strong adhesion might be needed, although other bridging polymers with lower pKa's could potentially be used instead of chitosan. Although the physiologic pH of skin trends to be slightly acidic, particularly in wound healing models, it is possible that skin pH may be adjusted through rinsing with warm water prior to gel application. Furthermore, most applications of adhesive materials in vivo will be with tissues of neutral pH. Under neutral and high pH, the formation of the chitosan interpenetrating network between the adherends is stable and non-reversible, likely as a significant decrease in pH would be needed to disturb the interchain H-bonds.

Several important factors influencing adhesion include properties of alginate (MW), tissue (pH), and chitosan (concentration, viscosity, degree of deacetylation, and MW). Although the main driver of adhesion with chitosan was the pH of the adherends, adhesion was significantly affected by chitosan concentration and MW, likely due to their effect on chitosan chain diffusion and interactions. Both high MW chitosan chains and concentrated polymer solutions have high viscosity due to chain-chain overlap, intermolecular interactions, and entanglements affecting the ability of the chains to diffuse into the permeable gel and tissue.^{[24]} Chitosan solutions of 1-2% (wt/vol) resulted in the highest adhesion energy, likely due to an optimal combination of viscosity and chain density. Small dilutions of chitosan in the presence of blood likely do not impact adhesion as the decreased chain density is offset by a lower viscosity and improved chitosan tissue penetration. Without wishing to be bound by the theory, diffusion and entanglement of the chitosan chains with the adherends play a key role. Thus, as demonstrated here, lower MW chitosan is expected to yield stronger topological adhesion because of the faster diffusion of low MW polymers. By using ultrapure chitosan which exhibits significantly lower viscosity while maintaining a high degree of deacetylation and medium molecular weight, much higher concentrations than previously possible with non-UP chitosan can be tested.^{[1]} Additionally, the range of chitosan MW tested in the previous study relied on non-UP materials and covered a narrower MW range. Regarding gel properties, the greatest impact on topological adhesion was the molecular weight of the alginate. Alginate molecular weight may increase the density of entanglements in the initial alginate/AAm solutions, especially since the formulation used is beyond the critical concentration of high MW alginate, ˜0.6 wt % (for 196 kDa alginate).^{[25]} A highly entangled hydrogel matrix likely facilitates chitosan chain entanglement within the matrix to form an interpenetrating network.^{[26]} This possibility is supported by the strong positive linear correlation between alginate MW and adhesion strength. Thus, it is demonstrated that by using high MW alginate in the dissipative matrix the adhesion can be significantly increased, to values even higher than what has been previously reported with a similar tough hydrogel system relying on covalent bond formation.

Chitosan diffusion and interpenetration into the permeable adherends seems a dominant factor in the adhesion mechanism as properties like MW and concentration, which affect chitosan diffusion, also resulted in a decrease in adhesion. Previous studies examining chitosan diffusion into tissue^{[1, 4]} have highlighted significant tissue penetration. These studies found that chitosan penetrates as deep as ˜25 um in both tendon and skin after 10 min^{[4]} and 1 h^{[1]} of compression, respectively. Additionally, chitosan has been shown to enhance transdermal drug permeation in various therapeutics by reversibly loosening intercellular tight junctions, leading to a widening of the paracellular routes and higher permeability while allowing faster diffusion of hydrophilic macromolecules.^{[24]} This enhanced tissue permeability likely contributes to the rapid penetration of chitosan chains into tissue, allowing for chain entanglement and rapid, robust topological adhesion.

As interpenetration and physical entanglement of chitosan chains is proposed to be the primary mechanism of adhesion, enhancing their diffusion would be expected to reduce the adhesion time. It has previously been demonstrated that dry hydrophilic polymeric materials can rapidly remove interfacial water and promote near instantaneous adhesion (˜5 s of compression) to tissues, but through the formation of covalent bonds.^{[13, 28]} This enhanced initial adhesion is likely in part due to a change in the toughness of the gel due to partial dehydration. The dry polymers show coupled hydration and swelling upon contact with water, due to their hydrophilicity and high diffusivity of water.^{[29]} Here, in the presence of a semi-dilute chitosan solution, dehydrated gels demonstrate rapid uptake of the solution.^{[30]} This process may be enhanced by the electrostatic attractions between the negatively charged carboxylic groups in the hydrogel matrix and the positively charged amines in chitosan. Here, dry gels achieved strong adhesion with just one minute of compression, with rapid initial swelling of the gel and uptake of the chitosan into the gel without the need of creating covalent bonds.

In conclusion, the findings of this study demonstrate a new approach to achieve strong and fast adhesion via secondary interactions between a hydrogel and tissue, and reveal factors and mechanisms involved in this adhesion. This approach reduces the need for covalent crosslinking components and could further enable the development of new improved bioadhesives for emerging applications. Also discovered was the ability of the chitosan film to release a drug or other molecule into a solution over time.

### REFERENCES

[1] J. Li. A. D. Celiz, J. Yang, Q. Yang, I. Wamala, W. Whyte, B. R. Seo, N. V. Vasilyev, J. J. Vlassak, Z. Suo, D. J. Mooney, Science 2017, 357, 378.
[2] G. M. Taboada, K. Yang. M. J. N. Pereira, S. S. Liu, Y. Hu. J. M. Karp, N. Artzi, Y. Lee, Nature Reviews Materials 2020.
[3] S. O. Blacklow, J. Li, B. R. Freedman, M. Zeidi, C. Chen, D. J. Mooney, Sci Adv 2019, 5, eaaw3963.
[4] B. R. Freedman, A. Kuttler, N. Beckmann, S. Nam, D. Kent, M. Schuleit, F. Ramazani, N. Accart, A. Rock, J. Li, M. Kurz, A. Fisch, T. Ullrich, M. W. Hast, Y. Tinguely, E. Weber, D. J. Mooney, Nature Biomedical Engineering 2021.
[5] S. Hu, X. Pei, L. Duan, Z. Zhu, Y. Liu, J. Chen, T. Chen, P. Ji, Q. Wan, J. Wang, Nat Commun 2021, 12, 1689; Z. M. Wright, B. D. Holt, S. A. Sydlik, J Mater Chem B 2017, 5, 7743.
[6] S. P. Lazow, D. F. Labuz, B. R. Freedman, A. Rock, D. Zurakowski, D. J. Mooney, D. O. Fauza, J Pediatr Surg 2020; B. R. Freedman, O. Uzun, N. M. M. Luna, A. Rock, C. Clifford, E. Stoler, G. Ostlund-Sholars, C. Johnson, D. J. Mooney, Adv Mater 2021, 33, e2008553; B. R. Freedman, D. J. Mooney, Adv Mater 2019, 31, e1806695; J. Deng, H. Yuk, J. Wu, C. E. Varela, X. Chen, E. T. Roche, C. F. Guo, X. Zhao, Nat Mater 2021, 20, 229.
[7] Z. Ma, G. Bao, J. Li, Adv Mater 2021, 33, e2007663.
[8] J. Y. Sun, X. Zhao, W. R. Illeperuma, O. Chaudhuri, K. H. Oh, D. J. Mooney, J. J. Vlassak, Z. Suo, Nature 2012, 489, 133.
[9] J. Li, W. R. Illeperuma, Z. Suo, J. J. Vlassak, ACS Macro Letters 2014, 3, 4.
[10] L. Ge, S. Chen, Polymers (Basel) 2020, 12; G. Bovone, O. Y. Dudaryeva, B. Marco-Dufort, M. W. Tibbitt, ACS Biomater Sci Eng 2021, 7, 4048.
[11] S. Nam, D. Mooney, Chem Rev 2021, 121, 11336.
[12] Y. Hong, F. Zhou, Y. Hua, X. Zhang, C. Ni, D. Pan, Y. Zhang, D. Jiang, L. Yang, Q. Lin, Y. Zou, D. Yu, D. E. Arnot, X. Zou, L. Zhu, S. Zhang, H. Ouyang, Nat Commun 2019, 10, 2060; O. Konuray, X. Fernandez-Francos, X. Ramis, A. Serra, Polymers (Basel) 2018, 10.
[13] H. Yuk, C. E. Varela, C. S. Nabzdyk, X. Mao, R. F. Padera, E. T. Roche, X. Zhao, Nature 2019, 575, 169.
[14] D. A. Wang, S. Varghese, B. Sharma, I. Strehin, S. Fermanian, J. Gorham, D. H. Fairbrother, B. Cascio, J. H. Elisseeff, Nat Mater 2007, 6, 385.
[15] C. B. Aakeroy, K. Epa, S. Forbes, N. Schultheiss, J. Desper, Chemistry 2013, 19, 14998; A. J. Kinloch, Journal of Materials Science 1980, 15, 25.
[16] J. Chen, D. Wang, L. H. Wang, W. Liu, A. Chiu, K. Shariati, Q. Liu, X. Wang, Z. Zhong, J. Webb, R. E. Schwartz, N. Bouklas, M. Ma, Adv Mater 2020, 32, e2001628.
[17] C. Cui, T. Wu, X. Chen, Y. Liu, Y. Li, Z. Xu, C. Fan, W. Liu, Advanced Functional Materials 2020, 30.
[18] J. Yang, R. Bai, B. Chen, S. Zuo, Advanced Functional Materials 2019, 27.
[19] J. Yang, R. Bai, J. Li, C. Yang, X. Yao, Q. Liu, J. J. Vlassak, D. J. Mooney, Z. Suo, ACS Appl Mater Interfaces 2019.
[20] J. Yang, R. Bai, Z. Suo, Adv Mater 2018, 30, e1800671.
[21] N. Pandey, L. F. Soto-Garcia, J. Liao, Z. Philippe, K. T. Nguyen, Y. Hong, Biomater Sci 2020, 8, 1240.
[22] N. Mati-Baouche, P. Elchinger, H. Baynast, G. Pierre, C. Delattre, P. Michaud, European Polymer Journal 2014, 60, 14.
[23] S. J. Frost, D. Mawad, M. J. Higgins, H. Ruprai, R. Kuchel, R. D. Tilley, S. Myers, J. M. Hook, A. Lauto, NPG Asia Materials 2016, 8, e280; A. Lauto, D. Mawad, M. Barton, A. Gupta, S. C. Piller, J. Hook, BioMedical Engineering OnLine 2010, 9, 47.
[24] B. H. Zimm, The Journal of Chemical Physics 1956, 24, 269; J. K. Hwang, H. H. Shin, Korea-Australia Rheology Journal 2000, 12, 5; P. E. Rouse, The Journal of Chemical Physics 1953, 21, 1272.
[25] C. A. Bonino, M. D. Krebs, C. D. Saquing, S. I. Jeong, K. L. Shearer, E. Alsberg, S. A. Khan, Carbohydrate Polymers 2011, 85, 111.
[26] A. Pragya, S. Mutalik, M. W. Younas, S.-K. Pang, P.-K. So, F. Wang, Z. Zheng, N. Noor, RSC Advances 2021, 11, 10710.
[27] H. E. Junginger, J. C. Verhoef, Pharmaceutical Science & Technology Today 1998, 1, 370.
[28] S. Singla, G. Amarpuri, N. Dhopatkar, T. A. Blackledge, A. Dhinojwala, Nature Communications 2018, 9, 1890.
[29] X. Mao, H. Yuk, X. Zhao, Journal of the Mechanics and Physics of Solids 2020, 137, 103863.
[30] R. R. Klossner, H. A. Queen, A. J. Coughlin, W. E. Krause, Biomacromolecules 2008, 9, 2947.
[31] H. J. Kong, D. Kaigler, K. Kim, D. J. Mooney, Biomacromolecules 2004, 5, 1720.
1.1. J. Li. A. D. Celiz, J. Yang, Q. Yang, I. Wamala, W. Whyte, B. R. Seo, N. V. Vasilyev, J. J. Vlassak, Z. Suo, D. J. Mooney, Tough adhesives for diverse wet surfaces. Science 357. 378-381 (2017).
2.1. S. O. Blacklow, J. Li, B. R. Freedman, M. Zeidi, C. Chen, D. J. Mooney, Bioinspired mechanically active adhesive dressings to accelerate wound closure. Sci Adv 5, eaaw3963 (2019).
3.1. S. P. Lazow, D. F. Labuz, B. R. Freedman, A. Rock, D. Zurakowski, D. J. Mooney, D. O. Fauza, A novel two-component, expandable bioadhesive for exposed defect coverage: Applicability to prenatal procedures. J Pediatr Surg. (2020).
4.1. B. R. Freedman, A. Kuttler, N. Beckmann, S. Nam, D. Kent, M. Schuleit, F. Ramazani, N. Accart, A. Rock, J. Li, M. Kurz, A. Fisch, T. Ullrich, M. W. Hast, Y. Tinguely, E. Weber, D. J. Mooney, Enhanced tendon healing by a tough hydrogel with an adhesive side and high drug-loading capacity. Nature Biomedical Engineering, (2021).
5.1. B. R. Freedman, O. Uzun, N. M. M. Luna, A. Rock, C. Clifford, E. Stoler, G. Ostlund- Sholars, C. Johnson, D. J. Mooney, Degradable and Removable Tough Adhesive Hydrogels. Adv Mater 33, e2008553 (2021).
6.1. G. Bovone, O. Y. Dudaryeva, B. Marco-Dufort, M. W. Tibbitt, Engineering Hydrogel Adhesion for Biomedical Applications via Chemical Design of the Junction. ACS Biomater Sci Eng 7, 4048-4076 (2021).
7.1. S. Nam, D. Mooney, Polymeric Tissue Adhesives. Chem Rev 121, 11336-11384 (2021).
8.1. J. Yang. R. Bai, Z. Suo, Topological Adhesion of Wet Materials. Adv Mater 30, e1800671 (2018).
9.1. J. Steck, J. Yang, Z. Suo, Covalent Topological Adhesion. ACS Macro Lett 8, 754-758 (2019).
10.1. J. A. C. Cruz, B. R. Freedman, M. Lee, C. Johnson, H. Ijaz, D. J. Mooney, Rapid Ultra-Tough Topological Tissue Adhesives. Adv Mater, e2205567 (2022).
11.1. P. E. Rouse, A Theory of the Linear Viscoelastic Properties of Dilute Solutions of Coiling Polymers. The Journal of Chemical Physics 21, 1272-1280 (1953).
12.1. H. Yuk, C. E. Varela, C. S. Nabzdyk, X. Mao, R. F. Padera, E. T. Roche, X. Zhao, Dry double-sided tape for adhesion of wet tissues and devices. Nature 575, 169-174 (2019).
13.1. X. Mao, H. Yuk, X. Zhao, Hydration and swelling of dry polymers for wet adhesion. Journal of the Mechanics and Physics of Solids 137, 103863 (2020).
14.1. J. Y. Sun, X. Zhao, W. R. Illeperuma, O. Chaudhuri, K. H. Oh, D. J. Mooney, J. J. Vlassak, Z. Suo, Highly stretchable and tough hydrogels. Nature 489, 133-136 (2012).
15.1. E. P. Chan, J. M. Karp, R. S. Langer, A “Self-Pinning” Adhesive Based on Responsive Surface Wrinkles. J Polym Sci B Polym Phys 49, 40-44 (2011).
16.1. Q. Li, P. Zhang, C. Yang, H. Duan, W. Hong, Switchable adhesion between hydrogels by wrinkling. Extreme Mechanics Letters 43, (2021).
17.1. M. Wisniewska, T. Urban, E. Grzadka, V. I. Zarko, V. M. Gun'ko, Comparison of adsorption affinity of polyacrylic acid for surfaces of mixed silica-alumina. Colloid Polym Sci 292, 699-705 (2014).
18.1. H. Yuk, T. Zhang, G. A. Parada, X. Liu, X. Zhao, Skin-inspired hydrogel-elastomer hybrids with robust interfaces and functional microstructures. Nat Commun 7, 12028 (2016).
19.1. L. Sherwood, Human Physiology: From Cells to Systems. (Brooks/Cole, Belmont, CA, 2010).
20.1. R. Simic, J. Mandal, K. Zhang, N. D. Spencer, Oxygen inhibition of free-radical polymerization is the dominant mechanism behind the “mold effect” on hydrogels. Soft Matter 17, 6394-6403 (2021).
21.1. Y. A. Meier, K. Zhang, N. D. Spencer, R. Simic, Linking Friction and Surface Properties of Hydrogels Molded Against Materials of Different Surface Energies. Langmuir 35, 15805-15812 (2019).
22.1. T. Narita, A. Hirai, J. Xu, J. P. Gong, Y. Osada, Substrate effects of gel surfaces on cell adhesion and disruption. Biomacromolecules 1, 162-167 (2000).
23.1. J. Yang, R. Bai, J. Li, C. Yang, X. Yao, Q. Liu, J. J. Vlassak, D. J. Mooney, Z. Suo, Design Molecular Topology for Wet-Dry Adhesion. ACS Appl Mater Interfaces, (2019).
24.1. A. K. S. Chandel, A. Shimizu, K. Hasegawa, T. Ito, Advancement of Biomaterial-Based Postoperative Adhesion Barriers. Macromol Biosci 21, e2000395 (2021).
25.1. H. J. Kong, D. Kaigler, K. Kim, D. J. Mooney, Controlling rigidity and degradation of alginate hydrogels via molecular weight distribution. Biomacromolecules 5, 1720-1727 (2004).

1. A tough adhesive comprising a hydrogel and a bridging polymer, wherein:
said hydrogel comprises a first polymer network and a second polymer network;
after contacting said hydrogel and said bridging polymer with a surface, said hydrogel is adhered to said surface via said bridging polymer, and an adhesion between said surface and said hydrogel is greater than or equal to 400 J/m² approximately 3 to about 10 minutes; and
said tough adhesive does not include a coupling agent.

2. The tough adhesive of claim 1, wherein said hydrogel is not covalently bound to said surface or said bridging polymer.

3. The tough adhesive of claim 1, wherein said first polymer network is a polyacrylamide polymer, said second polymer network is an alginate polymer, and said bridging polymer is a chitosan polymer.

4. The tough adhesive of claim 1, wherein said hydrogel is dried before application to said surface.

5. The tough adhesive of claim 4, wherein said adhesion between said surface and said hydrogel is greater than or equal to 1000 J/m² approximately about 1 minute after contacting said dehydrated hydrogel and said bridging polymer with said surface.

6. The tough adhesive of claim 3, wherein said alginate polymer has an average molecular weight of about 100 kDa to about 300 kDa, or wherein said alginate polymer has an average molecular weight of about 200 kDa to about 300 kDa.

7. The tough adhesive of claim 3, wherein said chitosan polymer has a molecular weight of about 100 kDa to about 600 kDa, or wherein said chitosan polymer has a molecular weight of about 150 kDa to about 250 kDa.

8. The tough adhesive of any claim 1, wherein said bridging polymer is a chitosan solution which comprises about 1% to about 2% (weight/volume) chitosan.

9. The tough adhesive of claim 1, wherein the surface is a tissue or an elastomer.

10. (canceled)

11. A method of applying said tough adhesive of claim 1, comprising the steps of:
adding said bridging polymer to said hydrogel;
compressing said hydrogel gel with said bridging polymer onto said surface, and optionally, wherein said hydrogel with said bridging polymer is compressed onto said surface for up to about 1 minute, or wherein said hydrogel with said bridging polymer is compressed onto said surface for up to about 10 minutes.

12. A method of applying said tough adhesive of claim 4, comprising the steps of:
drying said hydrogel;
adding said bridging polymer to said dried hydrogel;
compressing said dried hydrogel gel with said bridging polymer onto said surface, and optionally, wherein said hydrogel with said bridging polymer is compressed onto said surface for up to about 1 minute, or wherein said hydrogel with said bridging polymer is compressed onto said surface for up to about 10 minutes.

13. (canceled)

14. A tough adhesive comprising:
a hydrogel comprising a first polymer network and an optional second polymer network; and
a dried bridging polymer film;
wherein an adhesion between said hydrogel and said bridging polymer is greater than or equal to 150 J/m² approximately about 5 seconds after contacting said hydrogel with said dried bridging polymer film; and
the tough adhesive does not include a coupling agent.

15. The tough adhesive of claim 14, wherein said film is not covalently bound to said hydrogel.

16. The tough adhesive of claim 14, wherein said first polymer network is a polyacrylamide polymer, said second polymer network is an alginate polymer, and said dried bridging polymer film is a dried chitosan polymer film.

17. The tough adhesive of claim 14, wherein said hydrogel further comprises a polyethylene mesh.

18. The tough adhesive of claim 14, wherein a first portion of said hydrogel is adhered to a second portion of said hydrogel via said bridging polymer, and wherein an adhesion between said first and second portions of said hydrogel is greater than or equal to 150 J/m² approximately about 5 seconds after contacting said first portion of hydrogel and said dried bridging polymer film with said second portion of said hydrogel.

19. The tough adhesive of claim 14, further comprising a first and second hydrogel which each comprise a first polymer network and an optional second polymer network;
wherein said first hydrogel is adhered to said second hydrogel via said bridging polymer;
wherein an adhesion between said first and second hydrogels is greater than or equal to 150 J/m² approximately about 5 seconds after contacting said first hydrogel and said dried bridging polymer film with said second portion of said hydrogel, and optionally comprising the step of compressing said dried bridging polymer between said first and second hydrogels.

20. A method of applying said tough adhesive of claim 14, comprising the step of compressing said dried bridging polymer either between a first and a second portion of said hydrogel or directly onto said hydrogel.

21. (canceled)

22. The method of claim 20, wherein said hydrogel and said dried bridging polymer are compressed for up to about 5 seconds, or wherein said hydrogel and said dried bridging polymer are compressed for up to about 1 minute, or wherein said hydrogel and said dried bridging polymer are compressed for up to about 10 minutes.

23. The tough adhesive of claim 14, wherein an adhesion between said tough adhesive and an elastomer is greater than or equal to 150 J/m² approximately about 5 seconds after contacting said elastomer with said tough adhesive, and optionally wherein the elastomer is a poly(acrylate) elastomer.

24. (canceled)
